(19) United States
(12) Patent Application Publication
(10) Pub. No.: US 2010/0158850 A1
(43) Pub. Date: Jun. 24, 2010

(54) DENDRIMER BASED MODULAR PLATFORMS

(75) Inventors: James R. Baker, JR., Ann Arbor, MI (US); Mark M. Banaszak Holl, Ann Arbor, MI (US); Xue-min Cheng, Ann Arbor, MI (US); Baohua Huang, Ann Arbor, MI (US); Daniel McNemy, Ann Arbor, MI (US); Douglas G. Mullen, Ann Arbor, MI (US); Thommey Thomas, Dexter, MI (US)

Correspondence Address: Casimir Jones, S.C., 2275 DEMING WAY, SUITE 310, MIDDLETON, WI, 53562, US

(73) Assignee: The Regents of the University of Michigan, Ann Arbor, MI (US)

(21) Appl. No.: 12/645,081

(22) Filed: Dec. 22, 2009

Related U.S. Application Data
(60) Provisional application No. 61/140,480, filed on Dec. 23, 2008.

(51) Int. Cl. A61K 31/74; C08G 69/00

(52) U.S. Cl. 424/78.08; 528/310

(57) ABSTRACT

The present invention relates to novel therapeutic and diagnostic dendrimer based modular platforms (e.g., drug delivery platforms). In particular, the dendrimer based modular platforms are configured such that two or more dendrimers (e.g., PAMAM dendrimers) are coupled together (e.g., via a cycloaddition reaction) wherein each of the coupled dendrimers is functionalized (e.g., functionalized for targeting, imaging, sensing, and/or providing a therapeutic or diagnostic material and/or monitoring response to therapy). In some embodiments, the present invention provides dendrimer based modular platforms having coupled dendrimers (e.g., two or more coupled dendrimers) wherein each dendrimer is conjugated to one or more functional groups (e.g., therapeutic agent, imaging agent, targeting agent, triggering agent) (e.g., for specific targeting and/or therapeutic use of the dendrimer based modular platform). In some embodiments, the functional groups are conjugated to the dendrimers via a linker and/or a triggering agent. In addition, the present invention is directed to methods of synthesizing dendrimer based modular platforms, compositions comprising the dendrimer based modular platforms, as well as systems and methods utilizing the dendrimer based modular platforms (e.g., in diagnostic and/or therapeutic settings (e.g., for the delivery of therapeutics, imaging, and/or targeting agents (e.g., in disease (e.g., cancer) diagnosis and/or therapy, etc.)).

### CROSS-REFERENCE TO RELATED APPLICATIONS

The present application claims priority to pending U.S. Provisional Patent Application No. 61/140,480, filed Dec. 23, 2008, hereby incorporated by reference in its entirety.

### FEDERALLY SPONSORED RESEARCH OR DEVELOPMENT

This invention was made with government support under contract No. 5RO1CA119409 awarded by the National Institutes of Health. The government has certain rights in the invention.

### FIELD OF THE INVENTION

The present invention relates to novel therapeutic and diagnostic dendrimer based modular platforms (e.g., drug delivery platforms). In particular, the dendrimer based modular platforms are configured such that two or more dendrimers (e.g., PAMAM dendrimers) are coupled together (e.g., via a cycloaddition reaction) wherein each of the coupled dendrimers is functionalized (e.g., functionalized for targeting, imaging, sensing, and/or providing a therapeutic or diagnostic material and/or monitoring response to therapy). In some embodiments, the present invention provides dendrimer based modular platforms having coupled dendrimers (e.g., two or more coupled dendrimers) wherein each dendrimer is conjugated to one or more functional groups (e.g., therapeutic agent, imaging agent, targeting agent, triggering agent) (e.g., for specific targeting and/or therapeutic use of the dendrimer based modular platform). In some embodiments, the functional groups are conjugated to the dendrimers via a linker and/or a triggering agent. In addition, the present invention is directed to methods of synthesizing dendrimer based modular platforms, compositions comprising the dendrimer based modular platforms, as well as systems and methods utilizing the dendrimer based modular platforms (e.g., in diagnostic and/or therapeutic settings (e.g., for the delivery of therapeutics, imaging, and/or targeting agents (e.g., in disease (e.g., cancer) diagnosis and/or therapy, etc.)).

### BACKGROUND OF THE INVENTION

Cancer remains the number two cause of mortality in the United States, resulting in over 500,000 deaths per year. Despite advances in detection and treatment, cancer mortality remains high. New compositions and methods for the imaging and treatment (e.g., therapeutic) of cancer may help to reduce the rate of mortality associated with cancer.

Cancer is currently treated using a variety of modalities including surgery, radiation therapy and chemotherapy. The choice of treatment modality will depend upon the type, location and dissemination of the cancer. For example, many common neoplasms, such as colon cancer, respond poorly to available therapies.

For tumor types that are responsive to current methods, only a fraction of cancers respond well to the therapies. In addition, despite the improvements in therapy for many cancers, most currently used therapeutic agents have severe side effects. These side effects often limit the usefulness of chemotherapeutic agents and result in a significant portion of cancer patients without any therapeutic options. Other types of therapeutic initiatives, such as gene therapy or immunotherapy, may prove to be more specific and have fewer side effects than chemotherapy. However, while showing some progress in a few clinical trials, the practical use of these approaches remains limited at this time.

Despite the limited success of existing therapies, the understanding of the underlying biology of neoplastic cells has advanced. The cellular events involved in neoplastic transformation and altered cell growth are now identified and the multiple steps in carcinogenesis of several human tumors have been documented (See e.g., Isaacs, Cancer 70:1810 (1992)). Oncogenes that cause unregulated cell growth have been identified and characterized as to genetic origin and function. Specific pathways that regulate the cell replication cycle have been characterized in detail and the proteins involved in this regulation have been cloned and characterized. Also, molecules that mediate apoptosis and negatively regulate cell growth have been clarified in detail (Kerr et al., Cancer 73:2013 (1994)). It has now been demonstrated that manipulation of these cell regulatory pathways has been able to stop growth and induce apoptosis in neoplastic cells (See e.g., Cohen and Tohoku, Exp. Med., 168:351 (1992) and Fujiwara et al., J. Natl. Cancer Inst., 86:458 (1994)). The metabolic pathways that control cell growth and replication in neoplastic cells are important therapeutic targets.

Despite these impressive accomplishments, many obstacles still exist before these therapies can be used to treat cancer cells in vivo. For example, these therapies require the identification of specific pathophysiologic changes in an individual's particular tumor cells. This requires mechanical invasion (biopsy) of a tumor and diagnosis typically by in vitro cell culture and testing. The tumor phenotype then has to be analyzed before a therapy can be selected and implemented. Such steps are time consuming, complex, and expensive.

There is a need for treatment methods that are selective for tumor cells compared to normal cells. Current therapies are only relatively specific for tumor cells. Although tumor targeting addresses this selectivity issue, it is not adequate, as most tumors do not have unique antigens. Further, the therapy ideally should have several, different mechanisms of action that work in parallel to prevent the selection of resistant neoplasms. The therapy ideally should allow the physician to identify residual or minimal disease before and immediately after treatment, and to monitor the response to therapy. This is important since a few remaining cells may result in re-growth, or worse, lead to a tumor that is resistant to therapy. Identifying residual disease at the end of therapy (i.e., rather than after tumor regrowth) may facilitate eradication of the few remaining tumor cells.

Thus, an ideal therapy should have the ability to target a tumor, image the extent of the tumor (e.g., tumor metastasis) and identify the presence of the therapeutic agent in the tumor cells. Thus, therapies are needed that allows the physician to select therapeutic molecules based on the pathophysiologic abnormalities in the tumor cells, to document the response to the therapy, and to identify residual disease.

### SUMMARY

Modular dendrimer-based drug delivery platforms were designed during the course of development of embodiments for the present invention to improve upon existing limitations in single dendrimer systems. Using this modular strategy, biologically active platforms capable of, for example, targeting (e.g., receptor mediated targeting) and imaging (e.g., florescence imaging) were synthesized. Synthesis was accomplished through, for example, coupling a folic acid (FA) conjugated dendrimer with a fluorescein isothiocyanate (FITC) conjugated dendrimer. The two different dendrimer modules were coupled via the 1,3-dipolar cycloaddition reaction ('click' chemistry) between an alkyne moiety on the surface of the first dendrimer and an azide moiety on the second dendrimer. Two simplified model systems were also synthesized to develop appropriate click reaction conditions. Conjugates were characterized by ¹H NMR spectroscopy and NOESY. The FA-FITC modular platform was evaluated in vitro with a human epithelial cancer cell line (KB) and found to specifically target the over-expressed folic acid receptor.

The dendrimer-based modular systems of the present invention provide significant benefits over predecessor systems. For example, in using ‘click’ chemistry rather than, for example, oligonucleotide linking, the modular system are scaled up with far greater ease and at a substantially lower cost. Oligonucleotides are typically purchased in nano-gram quantities whereas the ‘click’ linkers are produced at the gram scale. Additionally, because the clicked dendrimers are covalently linked rather than joined via the hydrogen-bond base-pairing oligonucleotide bridge, the platform is less likely to become unlinked. This characteristic proves beneficial when attempting to isolate and characterize multi-module platforms.

Accordingly, in certain embodiments, the present invention provides compositions comprising a first dendrimer coupled with a second dendrimer. The compositions are not limited to a particular manner of coupling between the first and second dendrimers. In some embodiments, the coupling is a covalent attachment between the first dendrimer and the second dendrimer. In some embodiments, the covalent attachment is between an alkyne linker on the first dendrimer and an azide linker on the second dendrimer, or between an alkyne linker on the second dendrimer and an azide linker on the first dendrimer.

In some embodiments, the first dendrimer and second dendrimer each independently comprise at least one functional group such as, for example, a therapeutic agent, a targeting agent, a trigger agent, and an imaging agent.

The compositions are not limited to particular therapeutic agents. Examples of such therapeutic agents include, but are not limited to, the therapeutic agent is selected from the group consisting of a chemotherapeutic agent, an anti-oncogenic agent, an anti-angiogenic agent, a tumor suppressor agent, an anti-microbial agent, an expression construct comprising a nucleic acid encoding a therapeutic protein, a pain relief agent, a pain relief agent antagonist, an agent designed to treat an inflammatory disorder, an agent designed to treat an autoimmune disorder, an agent designed to treat inflammatory bowel disease, and an agent designed to treat inflammatory pelvic disease. In some embodiments, the agent designed to treat an inflammatory disorder includes, but is not limited to, an antirheumatic drug, a biologicals agent, a nonsteroidal anti-inflammatory drug, an analgesic, an immunomodulator, a glucocorticoid, a TNF-α inhibitor, an IL-1 inhibitor, and a metalloprotease inhibitor. In some embodiments, the antirheumatic drug includes, but is not limited to, leflunomide, methotrexate, sulfasalazine, and hydroxychloroquine;

wherein the biologicals agent is selected from the group consisting of rituximab, finfliximab, etanercept, adalimumab, and golimumab. In some embodiments, the nonsteroidal anti-inflammatory drug includes, but is not limited to, ibuprofen, celecoxib, ketoprofen, naproxen, piroxicam, and diclofenac. In some embodiments, the analgesic includes, but is not limited to, acetaminophen, and tramadol. In some embodiments, the immunomodulator includes but is not limited to anakinra, and abatacept. In some embodiments, the glucocorticoid includes, but is not limited to, prednisone, and methylprednisone. In some embodiments, the TNF-α inhibitor includes but is not limited to adalimumab, certolizumab pegol, etanercept, golimumab, and infliximab. In some embodiments, the autoimmune disorder and/or inflammatory disorder includes, but is not limited to, arthritis, psoriasis, lupus erythematosus, Crohn's disease, and sarcoidosis. In some embodiments, examples of arthritis include, but are not limited to, osteoarthritis, rheumatoid arthritis, septic arthritis, gout and pseudo-gout, juvenile idiopathic arthritis, psoriatic arthritis, Still's disease, and ankylosing spondylitis.

In some embodiments, the first dendrimer and/or the second dendrimer comprise at least one therapeutic agent conjugated with the first dendrimer and/or the second dendrimer via a trigger agent. The compositions are not limited to a particular trigger agent. In some embodiments, the trigger agent has a function selected from the group consisting of permitting a delayed release of a functional group from the dendrimer, permitting a constitutive release of the therapeutic agent from the dendrimer, permitting a release of a functional group from the dendrimer under conditions of acidosis, permitting a release of a functional group from a dendrimer under conditions of hypoxia, and permitting a release of the therapeutic agent from a dendrimer in the presence of a brain enzyme. Examples of trigger agents include, but are not limited to, an ester bond, an amide bond, an ether bond, an indoquinone, a nitroheterocyle, and a nitroimidazole.

The compositions are not limited to particular imaging agents. Examples of imaging agents include, but are not limited to, fluorescein isothiocyanate (FITC), 6-TAMARA, acridine orange, and cis-parinaric acid.

The compositions are not limited to particular targeting agent. Examples of targeting agents include, but are not limited to, an agent binding a receptor selected from the group consisting of CFTR, EGFR, estrogen receptor, FGR2, folate receptor, IL-2 receptor, and VEGFR; an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein; an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen; transferrin; and a synthetic tetanus toxin fragment.

The compositions are not limited to particular types of dendrimers. Examples of dendrimers include, but are not limited to, a polyamideamine (PAMAM) dendrimer, a polypropylamine (POPAM) dendrimer, and a PAMAM-POPAM dendrimer. In some embodiments, the dendrimer is a Baker-Huang PAMAM dendrimer. In some embodiments, at least one of the first dendrimer and/or the second dendrimer has a generation between 0 and 5. In some embodiments, at least one of the first dendrimer and/or the second dendrimer is at least partially acetylated.

In certain embodiments, the present invention provides methods for treating a disorder selected from the group consisting of any type of cancer or cancer-related disorder (e.g., tumor, a neoplasm, a lymphoma, or a leukemia), a neoplastic disease, osteoarthritis, rheumatoid arthritis, septic arthritis, gout and pseudo-gout, juvenile idiopathic arthritis, psoriatic arthritis, Still's disease, and ankylosing spondylitis, comprising administering to a subject suffering from the disorder a dendrimer based modular platform (e.g., a composition comprising a first dendrimer coupled with a second dendrimer, wherein the coupling is a covalent attachment between an alkyne linker on said first dendrimer and an azide linker on said second dendrimer).

In some embodiments, the composition is co-administered with an agent selected from the group consisting of an antirheumatic drug, a biologicals agent, a nonsteroidal anti-inflammatory drug, an analgesic, an immunomodulator, a glucocorticoid, a TNF-α inhibitor, an IL-1 inhibitor, and a metalloprotease inhibitor. In some embodiments, the antirheumatic drug is selected from the group consisting of leflunomide, methotrexate, sulfasalazine, and hydroxychloroquine. In some embodiments, the biologicals agent is selected from the group consisting of rituximab, finfliximab, etanercept, adalimumab, and golimumab. In some embodiments, the nonsteroidal anti-inflammatory drug is selected from the group consisting of ibuprofen, celecoxib, ketoprofen, naproxen, piroxicam, and diclofenac. In some embodiments, the analgesics is selected from the group consisting of acetaminophen, and tramadol. In some embodiments, the immunomodulator is selected from the group consisting of anakinra, and abatacept. In some embodiments, the glucocorticoid is selected from the group consisting of prednisone, and methylprednisone. In some embodiments, the TNF-α inhibitor is selected from the group consisting of adalimumab, certolizumab pegol, etanercept, golimumab, and infliximab.

In some embodiments, the composition is co-administered with an anti-cancer agent, a pain relief agent, and/or a pain relief agent antagonist.

In some embodiments, the neoplastic disease includes, but is not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-0, human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In certain embodiments, the present invention provides methods for coupling a first dendrimer with a second dendrimer, comprising exposing the first dendrimer to the second dendrimer under conditions such that covalent attachment occurs between an alkyne linker on the first dendrimer and an azide linker on the second dendrimer. In some embodiments, the first dendrimer and second dendrimer each independently comprise at least one functional group selected from the group consisting of a therapeutic agent, an imaging agent, and a targeting agent. In some embodiments, the first dendrimer and/or the second dendrimer is selected from the group consisting of a polyamideamine (PAMAM) dendrimer, a polypropylamine (POPAM) dendrimer, and a PAMAM-POPAM dendrimer. In some embodiments, the coupling occurs via a cycloaddition reaction between the first dendrimer and the second dendrimer.

In certain embodiments, the present invention provides compositions comprising a first dendrimer coupled with a second dendrimer. The compositions are not limited to a particular manner of coupling. In some embodiments, the coupling is a covalent attachment between an alkyne linker on the first dendrimer and an azide linker on the second dendrimer. In some embodiments, the first dendrimer and second dendrimer each independently comprise at least one functional group selected from the group consisting of a therapeutic agent, an imaging agent, and a targeting agent.

The compositions are not limited to a particular therapeutic agent. Examples of therapeutic agents include, but are not limited to, chemotherapeutic agents, anti-oncogenic agents, anti-angiogenic agents, tumor suppressor agents, anti-microbial agents, expression constructs comprising a nucleic acid encoding a therapeutic protein, pain relief agents, pain relief agent antagonists, agents designed to treat arthritis, agents designed to treat inflammatory bowel disease, agents designed to treat an autoimmune disease, and agents designed to treat inflammatory pelvic disease.

In some embodiments, the functional group is attached with the first dendrimer and/or the second dendrimer via a trigger agent. The present invention is not limited to particular type or kind of trigger agent. In some embodiments, the trigger agent is configured to have a function such as, for example, a) a delayed release of the functional group from the first dendrimer and/or the second dendrimer, b) a constitutive release the therapeutic agent from the first dendrimer and/or the second dendrimer, c) a release of the functional group from the first dendrimer and/or the second dendrimer under conditions of acidosis, d) a release of the functional group from the first dendrimer and/or the second dendrimer under conditions of hypoxia, and e) a release of the therapeutic agent from the first dendrimer and/or the second dendrimer in the presence of a brain enzyme. Examples of trigger agents include, but are not limited to, an ester bond, an amide bond, an ether bond, an indoquinone, a nitroheterocyle, and a nitroimidazole. In some embodiments, the trigger agent is attached with the first dendrimer and/or the second dendrimer via a linker. The present invention is not limited to a particular type or kind of linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

The compositions are not limited to a particular type or kind of imaging agent. In some embodiments, the imaging agent comprises fluorescein isothiocyanate or 6-TAMARA.

The compositions are not limited to a particular type or kind of targeting agent. In some embodiments, the targeting agent is configured to target the composition to cancer cells. In some embodiments, the targeting agent comprises folic acid. In some embodiments, the targeting agent binds a receptor selected from the group consisting of CFTR, EGFR, estrogen receptor, FGR2, folate receptor, IL-2 receptor, VEGFR. In some embodiments, the targeting agent comprises an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein. In some embodiments, the targeting agent comprises an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen. In some embodiments, the targeting agent is configured to permit the composition to cross the blood brain barrier. In some embodiments, the targeting agent is transferrin. In some embodiments, the targeting agent is configured to permit the composition to bind with a neuron within the central nervous system. In some embodiments, the targeting agent is a synthetic tetanus toxin fragment. In some embodiments, the synthetic tetanus toxin fragment comprises an amino acid peptide fragment. In some embodiments, the amino acid peptide fragment is HLNILSTLWKYR.

The compositions are not limited to particular types or kinds of dendrimers. Examples of dendrimers include, but are not limited to, a polyamideamine (PAMAM) dendrimer, a polypropylamine (POPAM) dendrimer, and a PAMAM-POPAM dendrimer. In some embodiments, dendrimer is at least partially acetylated.

In certain embodiments, the present invention provides methods of treating cancer cells comprising exposing the cancer cells to at least one composition comprising a first dendrimer coupled with a second dendrimer, wherein the coupling is a covalent attachment between an alkyne linker on the first dendrimer and an azide linker on the second dendrimer, wherein first dendrimer comprises a therapeutic agent, wherein the second dendrimer comprises a targeting agent. In some embodiments, the targeting agent is configured to target the composition to cancer cells. In some embodiments, the cancer cells are selected from the group consisting of in vivo, in vitro, and ex vivo. In some embodiments, the cancer cells are in a human.

In some embodiments, the targeting agent comprises folic acid. In some embodiments, the targeting agent binds a receptor selected from the group consisting of CFTR, EGFR, estrogen receptor, FGR2, folate receptor, IL-2 receptor, VEGFR. In some embodiments, the targeting agent comprises an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein. In some embodiments, the targeting agent comprises an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA 125 antigen from cystadenocarcinoma, DF3 antigen from human breast carcinoma, and p97 antigen of human melanoma, carcinoma or orosomucoid-related antigen.

In some embodiments, the first dendrimer and/or the second dendrimer comprises an imaging agent. In some embodiments, the imaging agent comprises fluorescein isothiocyanate or 6-TAMARA.

In some embodiments, applicable therapeutic agents include, but are not limited to, a chemotherapeutic agent, an anti-oncogenic agent, an anti-angiogenic agent, a tumor suppressor agent, an anti-microbial agent, an expression construct comprising a nucleic acid encoding a therapeutic protein.

In some embodiments, the therapeutic agent is attached with the first dendrimer via a trigger agent. In some embodiments, the trigger agent is configured to delay release of the functional group from the first dendrimer and/or the second dendrimer, and/or to constitutively release the therapeutic agent from the first dendrimer and/or the second dendrimer. Examples of trigger agents include, but are not limited to, an ester bond, an amide bond, an ether bond, an indoquinone, a nitroheterocyle, and a nitroimidazole. In some embodiments, the trigger agent is attached with the first dendrimer via a linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

In some embodiments, the first dendrimer and/or the second dendrimer is selected from the group consisting of a polyamideamine (PAMAM) dendrimer, a polypropylamine (POPAM) dendrimer, and a PAMAM-POPAM dendrimer. In some embodiments, the first dendrimer and/or the second dendrimer is at least partially acetylated. In some embodiments, the dendrimer is a Baker-Huang PAMAM dendrimer.

In certain embodiments, the present invention provides methods of coupling a first dendrimer with a second dendrimer, comprising exposing the first dendrimer to the second dendrimer under conditions such that covalent attachment occurs between an alkyne linker on the first dendrimer and an azide linker on the second dendrimer. In some embodiments, the first dendrimer and the second dendrimer each independently comprise at least one functional group selected from the group consisting of a therapeutic agent, an imaging agent, and a targeting agent. In some embodiments, the first dendrimer and/or the second dendrimer is a polyamideamine (PAMAM) dendrimer, a polypropylamine (POPAM) dendrimer, or a PAMAM-POPAM dendrimer. In some embodiments, the first dendrimer and/or the second dendrimer is at least partially acetylated. In some embodiments, the coupling occurs via a cycloaddition reaction (e.g., a 1,3-dipolar cycloaddition reaction) between the first dendrimer and the second dendrimer.

### DESCRIPTION OF THE DRAWINGS

FIG. 1 shows a) NOESY of the small-molecule model system after the ‘click’ reaction (4). NOE cross-peaks between triazole related protons (b, c, e, f, and g) are labeled. b) NOESY of the model dendrimer system after the ‘click’ reaction (7). NOE cross-peaks for the triazole related protons are similarly labeled. The cross-peaks in the 2D spectra reveal proton chemical shifts for the several of the triazole related protons that are otherwise obscured by overlapping dendrimer peaks. c) Chemical structure and proton labels for the clicked small molecule model system (4) and the clicked dendrimer model system (7). The G5 PAMAM dendrimer is represented by a gray sphere.

FIG. 2 shows proton NMR spectra of the small-molecule model system and model dendrimer system both pre- and post-‘click’ reaction. An up-field shift is observed for aromatic proton g as a result of the ‘click’ reaction. a) Spectrum of the small molecule model system before the ‘click’ reaction (2a and 3b), taken in CDCl₃. Proton g has a chemical shift of 6.85 ppm. b) Spectrum of the small molecule model system after the ‘click’ reaction (4), taken in CDCl₃. As a result of the ‘click’ reaction, g has experienced an up-field change to 6.78 ppm. c) Spectrum of the model dendrimer system before the ‘click’ reaction (5 and 6), taken in D₂O. Proton g overlaps proton b at 6.90 ppm. d) Spectrum of the model dendrimer system after the ‘click’ reaction (7), taken in D₂O, Similar to the small molecule model system, proton g experiences an up-field change as a result of the ‘click’ reaction. In the model dendrimer system, the new chemical shift is 6.74 ppm.

FIG. 3 shows synthetic scheme for the model dendrimer system (7). This simplified modular platform was developed to assist with the spectroscopic characterization of the folic acid targeted dendrimer system. The G5 dendrimer, used in this study, had an average of 112 end groups as determined by GPC and potentiometric titration.

FIG. 4 shows synthetic scheme for the folic acid targeted modular dendrimer-based platform (15). A module possessing a terminal alkyne moiety and Folic Acid (13) is coupled to a second module possessing a terminal azide moiety and FITC (14) via the Cu-catalyzed 1,3-dipolar cycloaddition reaction. Both modules were fully acetylated to avoid non-specific cellular interactions.

FIG. 5 shows uptake of the fluorescent modular targeted dendrimer platform in KB cells as measured by Flow Cytometry. a) Uptake FA_{3.5}-G5-Ac₁₀₇-L-G5Ac₁₀₆-FITC_{3.2} (15). b) Uptake of G5-Ac₁₀₆-Azide_{2.5}-FITC_{3.2} (14) is not observed for 30 nM, 100 nM, and 300 nM. Very minimal uptake of this un-targeted module is observed at 1000 nM. c) Similarly, no uptake is observed for an uncoupled mixture of G5-Ac₁₀₆-Azide_{2.5}-FITC_{3.2} (14) and G5-Ac₁₀₇-Alkyne₁₀₆-FA_{3.5} (13). d) Uptake of FA_{3.5}-G5-Ac₁₀₇-L-G5Ac₁₀₆-FITC_{3.2} (15) is successfully blocked using a 20 fold excess of free folic acid. e) Uptake of FA_{3.5}-G5-Ac₁₀₇-L-G5Ac₁₀₆-FITC_{3.2} (15) is also successfully blocked using a 20 fold excess (with respect to the folic acid content) of G5-Ac₁₀₇-Alkyne_{1.6}-FA_{3.5} (13). f) Summary of mean fluorescence values for a-e. Uptake of FA_{3.5}-G5-Ac₁₀₇-L-G5Ac₁₀₆-FITC_{3.2} (15) is displayed in blue. Uptake of the targeted platform (15) blocked by a 20 fold excess of G5-Ac₁₀₇-Alkyne_{1.6}-FA_{3.5} (13) is shown in orange. Uptake of the targeted platform (15) blocked by a 20 fold excess of free folic acid can be found in green. Uptake of a mixture of G5-Ac₁₀₆-Azide_{2.5}-FITC_{3.2} (14) and G5-Ac₁₀₆-Alkyne_{1.6}-FA_{3.5} (13) can be found in teal. Finally, uptake of G5-Ac₁₀₆-Azide_{2.5}-FITC_{3.2} (14) can be found in purple. Error bars. indicate standard deviation as computed from half-peak coefficient of variation (HPCV) values.

FIG. 6A-Q shows NMR spectra for various dendrimer conjugates.

FIG. 7 shows binding and uptake of the fluorescent modular targeted dendrimer platform and controls in KB cells as measured by Flow Cytometry. Uptake of FA_{3.5}-G5-Ac₁₀₇-L-G5Ac₁₀₆-FITC_{3.2} (15) is displayed in blue. Mean fluorescence values for the un-targeted platform G5-Ac_{110.7}-L-G5-Ac₁₀₆-FITC_{3.2} (18) is shown in green. Last, mean fluorescence values for the imaging module G5-Ac₁₀₆-Azide_{2.5}-FITC_{3.2} (14) can be found in purple. Error bars indicate standard deviation as computed from half-peak coefficient of variation (HPCV) values.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

As used herein, the term “nanodevice” or “nanodevices” refer, generally, to compositions comprising dendrimers of the present invention. As such, a nanodevice may refer to a composition comprising a dendrimer of the present invention that may contain one or more ligands, linkers, and/or functional groups (e.g., a therapeutic agent, a targeting agent, a trigger agent, an imaging agent) conjugated to the dendrimer.

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076, herein incorporated by reference in its entirety). Similarly, the conjugate may comprise a cleavable linkage present in the linkage between the dendrimer and functional group, or, may comprise a cleavable linkage present in the polymer itself (See, e.g., U.S. Pat. App. Nos. 20050158273 and 20050181449, each of which is herein incorporated by reference in its entirety).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterizied. For example, an inhibitor can be selected from the group comprising, but not limited to, those found in U.S. Pat. No. 6,011,021, herein incorporated by reference in its entirety.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “click chemistry” refers to chemistry tailored to generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362; each herein incorporated by reference in its entirety).

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino) pyridine, or dicyclohexylcarbodiimide and 4-(dimethylamino) pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “ligand” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound (e.g., triazine scaffold) intervenes. In preferred embodiments, ligands have functional utility for specific applications, e.g., for therapeutic, targeting, imaging, or drug delivery function(s). The terms “ligand”, “conjugate”, and “functional group” may be used interchangeably.

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants.

As used herein, the term “amino alcohol” or “amino-alcohol” refers to any organic compound containing both an amino and an aliphatic hydroxyl functional group (e.g., which may be an aliphatic or branched aliphatic or alicyclic or hetero-alicyclic compound containing an amino group and one or more hydroxyl(s)). The generic structure of an amino alcohol may be expressed as NH₂—R—(OH)ₘ wherein m is an integer, and wherein R comprises at least two carbon molecules (e.g., at least 2 carbon molecules, 10 carbon molecules, 25 carbon molecules, 50 carbon molecules).

As used herein, the term “Baker-Huang dendrimer” or “Baker-Huang PAMAM dendrimer” refers to a dendrimer comprised of branching units of structure:

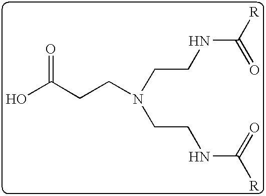

wherein R comprises a carbon-containing functional group (e.g., CF₃). In some embodiments, the branching unit is activated to its HNS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in Scheme 2). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines. In preferred embodiments, a Baker-Huang dendrimer is synthesized by convergent synthesis methods.

### DETAILED DESCRIPTION OF THE INVENTION

The high toxicity of conventional cytotoxic anti-cancer drugs often forces these agents to be given at sub-optimal dosages and this can result in treatment failure (see, e.g., Allen, T. M., Nature Reviews Cancer 2002, 2, (10), 750-763; herein incorporated by reference in its entirety). To resolve this problem, delivery platforms that can discriminate between healthy and malignant cells have been developed (see, e.g., Allen, T. M., Nature Reviews Cancer 2002, 2, (10), 750-763; Peer, D., et al., Nature Nanotechnology 2007, 2, 751-760; each herein incorporated by reference in their entireties). Generally, targeted therapeutic delivery platforms consist of three different components: a targeting component comprised of targeting ligands with affinities for molecules expressed on cancer cells; a payload consisting of drug and/or imaging agents; and a nano-scale structure to which the targeting and payload moieties are attached. This platform targeting of anti-cancer drugs with cancer cell-specific ligands can dramatically improve a drug's therapeutic index. Conjugating multiple targeting ligands to a single platform molecule further increases the potential for specific targeting of cancer cells by allowing the possibility of multivalent interactions (see, e.g., Hong, S., et al., Chemistry & Biology 2007, 14, (1), 105-113; Mammen, M., et al., Angewandte Chemie-International Edition 1998, 37, (20), 2755-2794; each herein incorporated by reference in their entireties).

The structural design of these types of delivery platforms is critical to the success of the delivery device. Numerous classes of targeted drug delivery platforms have been developed that potentially meet the requirements needed to combine targeting ligands, imaging agents, and drug molecules together to deliver the therapeutic payload to a desired location in the body. These include drug-target conjugates, linear polymers, lipid-based carriers (liposomes and micelles), carbon nanotubes, inorganic nanoparticles, and dendrimers. Several of these different delivery platforms are progressing towards or through clinical trials for cancer treatments with promising results (see, e.g., Peer, D., et al., Nature Nanotechnology 2007, 2, 751-760; herein incorporated by reference in its entirety). Each approach, however, is not without limitations and the potential for widespread application of these platforms in their present design is unclear.

Dendrimer-based platforms have a unique branching structure which results in exceptionally high degrees of monodispersity and well defined terminal groups that provide the ability to form soluble conjugates containing multiple copies of hydrophobic drug and/or targeting molecules. The compact, branched structures appear to enhance the ability of the targeting molecules to interact in a fashion conducive to multivalent binding to cell membrane receptors (see, e.g., Hong, S., et al., Chemistry & Biology 2007, 14, (1), 105-113; herein incorporated by reference in its entirety). The dendrimer's small size enables efficient diffusion across the vascular endothelium to find tumors and also allows the rapid clearance of these molecules from the blood stream. This clearance avoids potential long-term toxicities and reduces the necessity of a rapidly-degradable platform. The most widely used dendrimer in biomedical applications, poly(amidoamine) (PAMAM), is non-immunogenetic and non-toxic once the surface primary amines have been modified (see, e.g., Majoros, I. J., et al., Macromolecules 2003, 36, (15), 5526-5529; Hong, S. P., et al., Bioconjugate Chemistry 2004, 15, (4), 774-782; Lee, C. C., et al., Nature Biotechnology 2005, 23, (12), 1517-1526; Svenson, S., et al., Advanced Drug Delivery Reviews 2005, 57, (15), 2106-2129; S. P. Hong, et al., Bioconjug. Chem. 17(3) (2006) 728-734; P. R. Leroueil, Acc. Chem. Res. 40(5) (2007) 335-342; each herein incorporated by reference in their entireties). There have been numerous, recent examples describing the development of dendrimer-based targeted delivery systems using a wide variety of targeting ligands including monoclonal antibodies (see, e.g., Thomas, T. P., et al., Biomacromolecules 2004, 5, (6), 2269-2274; Patri, A. K., et al., Bioconjugate Chemistry 2004, 15, (6), 1174-1181; Shukla, R., et al., Bioconjugate Chemistry 2006, 17, (5), 1109-1115; Wu, G., et al., Molecular Cancer Therapeutics 2006, 5, (1), 52-59; Wu, G., et al., Bioconjugate Chemistry 2004, 15, (1), 185-194; Backer, M. V., et al., Molecular Cancer Therapeutics 2005, 4, (9), 1423-1429; each herein incorporated by reference in their entireties), peptides (see, e.g., Shukla, R., et al., Chemical Communications 2005, (46), 5739-5741; herein incorporated by reference in its entirety), T-antigens (see, e.g., Sheng, K. C., et al., European Journal of Immunology 2008, 38, 424-436; Baek, M. G., et al., Bioorganic & Medicinal Chemistry 2002, 10, (1), 11-17; Taite, L. J., et al., Journal of Biomaterials Science-Polymer Edition 2006, 17, (10), 1159-1172; each herein incorporated by reference in their entireties), and folic acid (see, e.g., Kono, K., et al., Bioconjugate Chemistry 1999, 10, (6), 1115-1121; Shukla, S., et al., Bioconjugate Chemistry 2003, 14, (1), 158-167; Majoros, I. J., et al., Biomacromolecules 2006, 7, (2), 572-579; Thomas, T. P., et al., Journal of Medicinal Chemistry 2005, 48, (11), 3729-3735; Myc, A., et al., Anti-Cancer Drugs 2008, 19, 143-149; Majoros, I. J., et al., Journal of Medicinal Chemistry 2005, 48, (19), 5892-5899; Kukowska-Latallo, J. F., et al., Cancer Research 2005, 65, (12), 5317-5324; Myc, A., et al., Biomacromolecules 2007, 8, 2986-2989; Myc, A., et al., Biomacromolecules 2007, 8, (1), 13-18; Landmark, K. J., et al., ACS Nano 2008, 2, (4), 773-783; each herein incorporated by reference in their entireties).

Despite the success of these dendrimer-based platforms, this approach has several challenges associated with its implementation. First, the synthesis of dendrimers with different functional groups for targeted delivery (including targeting, drug, and imaging agents) requires a laborious chemical process that is unique for each different molecular combination. Second, the carrying capacity of a single dendrimer, although significantly better than other types of delivery platforms, is finite due to limits in surface molecule density and solubility. This becomes a problem when one attempts to conjugate multiple copies of the target, drug, and/or dye molecules to the same dendrimer. Third, significant increases in heterogeneity occur with the conjugation of each additional molecule to a single dendrimer platform due to the stochastic nature of these chemical reactions (see, e.g., D. G. Mullen, Bioconjug. Chem. 19(9) (2008) 1748-1752; herein incorporated by reference in its entirety). This has limited the flexibility of these systems.

To address the drawbacks of the single dendrimer platforms, several groups have sought to apply modular design concepts to dendrimer systems (see, e.g., Y. S. Choi, et al., Nano Lett. 4(3) (2004) 391-397; Y. Choi, Chem. Biol. 12(1) (2005) 35-43; C.R. DeMattei, Nano Lett. 4(5) (2004) 771-777; Y. Choi, Nanostructured Supramolecular Arrays Based on Dendrimers Using DNA: Design, Synthesis and Biological Evaluation. Biomed. Eng. (NY). Vol. Ph.D. Dissertation, University of Michigan, Ann Arbor, Mich., 2005, p. 191; J. W. Lee, Bioconjug. Chem. 18(2) (2007) 579-584; J. W. Lee, J. Polym. Sci., Part A: Polym. Chem. 46 (2008) 1083-1097; J. W. Lee, Macromolecules 39(6) (2006) 2418-2422; J. W. Lee, Tetrahedron 62(5) (2006) 894-900; P. Wu, Chem. Commun. (46) (2005) 5775-5777; P. Goyal, Chem. Eur. J. 13 (2007) 8801-8810; K. Yoon, Org. Lett. 9(11) (2007) 2051-2054; each herein incorporated by reference in their entireties). In an effort to address these drawbacks of the single dendrimer platform, the present invention provides systems and methods applying, for example, modular design concepts to the dendrimer system. A basic premise of this new design is to use dendrimers or dendrons as modular units and combine different modules together to create a multi-module platform. Multi-functional platforms can be generated by combining different modules through a universal coupling mechanism. Benefits of this design are two fold: First, segregating each functional group (drug/target/dye) to a different dendrimer module avoids the need to develop a new orthogonal coupling chemistry for each new combination of functional groups. This advantage should not be underestimated. Significant time is spent developing new orthogonal coupling strategies for desired functional combinations because many of the component drug molecules and targeting ligands (e.g., Taxol and RGD) are susceptible to a loss of activity due to undesired cross reactions as well as degradation by hydrolysis. The second benefit of the modular strategy is a greater carrying capacity of the delivery platform because the different functional molecules are localized on separate dendrimer units and the water solubilizing dendrimer backbone is effectively double the mass of the single dendrimer.

Oligonucleotide self-assembly has been used to link both dendron and dendrimer modular units. Choi and co-workers demonstrated this strategy by using two complementary oligonucleotides to link two PAMAM dendrimers together (see, e.g., Y. S. Choi, et al., Nano Lett. 4(3) (2004) 391-397; Y. Choi, Chem. Biol. 12(1) (2005) 35-43; each herein incorporated by reference in their entireties). DeMattie, Huang, and Tomalia used the same method to connect two un-functionalized PAMAM dendrons together (see, e.g., C. R. DeMattei, et al., Nano Lett. 4(5) (2004) 771-777; herein incorporated by reference in its entirety). Characterization of these systems was accomplished by gel electrophoresis, AFM, MALDI, and UV/vis due to the small synthetic scales employed. For the dendrimer-based system, isolated dendrimer samples were not obtained, rather the samples were generated in solution. Choi et al. did demonstrate biological functionality of the modular system in a cell culture assay (see, e.g., Y. Choi, et al., Chem. Biol. 12(1) (2005) 35-43; herein incorporated by reference in its entirety) and an in vivo model (see, e.g., Y. Choi, Nanostructured Supramolecular Arrays Based on Dendrimers Using DNA: Design, Synthesis and Biological Evaluation. Biomed. Eng. (NY). Vol. Ph.D. Dissertation, University of Michigan, Ann Arbor, Mich., 2005, p. 191; herein incorporated by reference in its entirety).

The use of ‘click’ chemistry to create dendritic modular systems has mainly involved dendrons. ‘Click’ chemistry is a particularly attractive coupling method because it can be performed with a wide variety of solvent conditions including aqueous environments. The stable triazole ring bridge, resulting from coupling alkyne with azide moieties, is frequently achieved at near quantitative yields and is considered to be biologically stable (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; P. Wu, et al., Aldrichimica Acta 40(1) (2007) 7-17; each herein incorporated by reference in their entireties). Furthermore, the ‘click’ coupling chemistry is orthogonal to the coupling chemistries typically used to attach functional groups to the dendrimer. In particular, the synthesis of multi-module platforms using both un-functionalized PAMAM dendrons (see, e.g., Lee, J. W.; et al., Bioconjugate Chemistry 2007, 18, (2), 579-584; Lee, J. W.; et al., Journal of Polymer Science Part a—Polymer Chemistry 2008, 46, 1083-1097; J. W. Lee, et al., Macromolecules 39(6) (2006) 2418-2422; each herein incorporated by reference in their entireties) as well as un-functionalized Frechet-type dendrons (see, e.g., Lee, J. W.; et al., Tetrahedron 2006, 62, (5), 894-900; herein incorporated by reference in its entirety) for each of the modules has been demonstrated. In all of these systems, the focal point of the dendron possessed either the azide or alkyne moiety. In addition, a 2,2-bis(hydroxymethyl)propionic acid (bis-MPA) based asymmetric modular dendron with 16 mannose units and 2 coumarin chromaphores has been demonstrated, and binding in a hemagglutination assay was shown (see, e.g., Wu, P.; et al., Chemical Communications 2005, (46), 5775-5777; herein incorporated by reference in its entirety). In addition, a poly(amine) dendrimer possessing a single aldehyde or azide moiety on the dendrimer periphery has been developed and shown to be capable of orthogonal functionalization by small molecule functional groups (see, e.g., Goyal, P.; et al., Chemistry—a European Journal 2007, 13, 8801-8810; Yoon, K., et al., Organic Letters 2007, 9, (11), 2051-2054; each herein incorporated by reference in their entireties). None of the described systems, however, have successfully used click chemistry to couple dendrimers.

The present invention provides compositions and related methods providing such compositions. Indeed, the present invention provides a new modular platform based upon, for example, clicking together dendrimers (e.g., generation 5 PAMAM dendrimers) optionally containing an agent (e.g., a targeting agent (e.g., the targeting agent folic acid (Compound 13)) (e.g., the dye fluorescein isothiocyanate (FITC) (Compound 14), a therapeutic agent, an imaging agent). In experiments conducted during the course of development of embodiments for the present invention, modular platforms based upon clicking together generation 5 PAMAM dendrimers containing either the targeting agent folic acid or the dye FITC (Compound 15) were synthesized. The systems were characterized by ¹H NMR spectroscopy and Nuclear Overhauser Effect spectroscopy (NOESY). The linking of two dendrimer-based modules together was achieved by first conjugating one of the dendrimer modules with an alkyne linker (2b) and conjugating the second dendrimer module with an azide linker (3c). The dose-dependent uptake of the clicked FA-FITC modules into KB cells was studied by flow cytometry and the ability of this modular system to specifically target folic acid expressing cells was verified. °

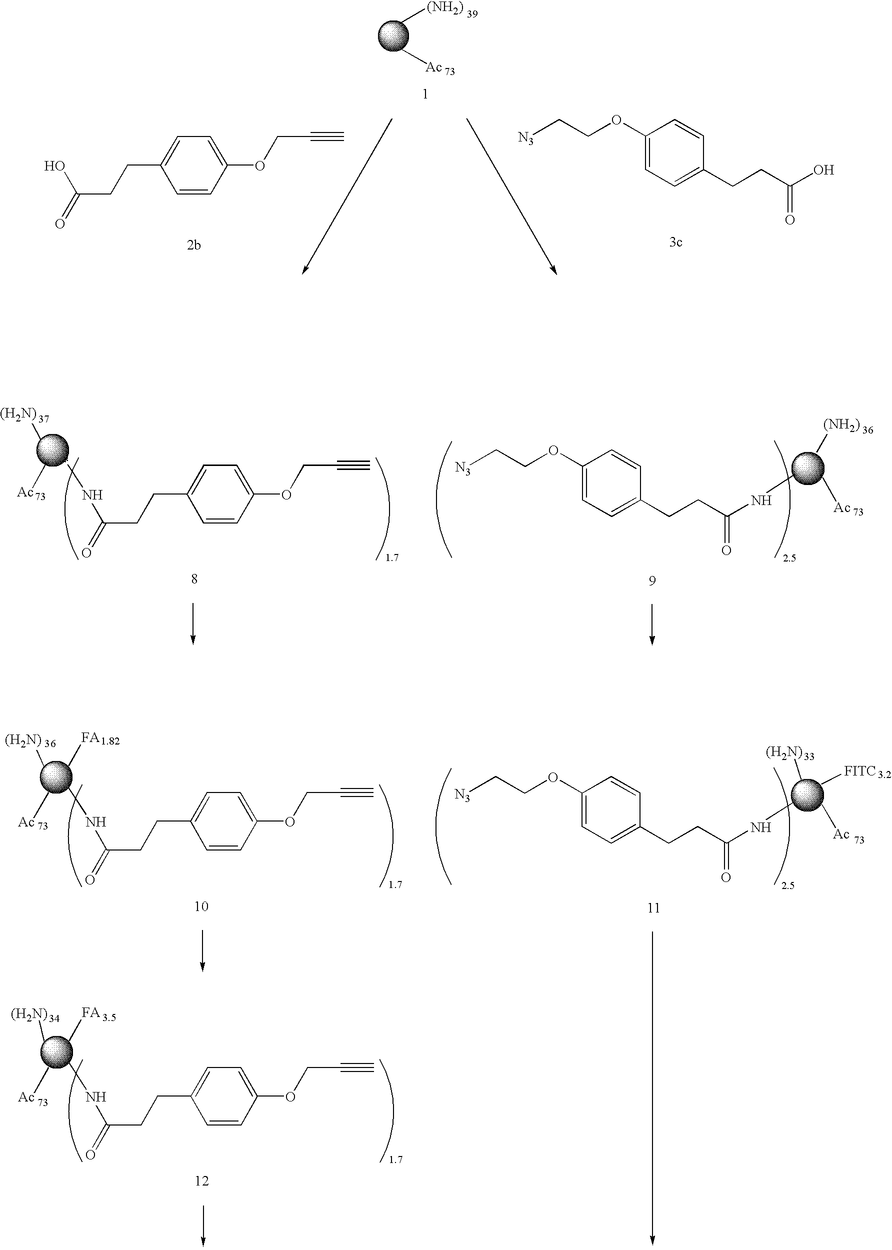

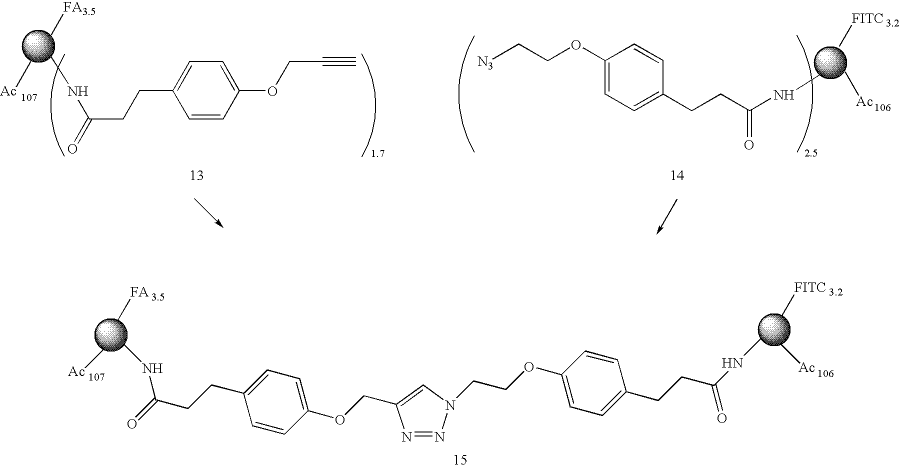

The dendrimer-based modular systems of the present invention provide significant benefits over predecessor systems. For example, in using ‘click’ chemistry rather than oligonucleotide linking, the modular system are scaled up with far greater ease and at a substantially lower cost. Oligonucleotides are typically purchased in nano-gram quantities whereas the ‘click’ linkers are produced at the gram scale. Additionally, because the clicked dendrimers are covalently linked rather than joined via the hydrogen-bond base-pairing oligonucleotide bridge, the platform is less likely to become unlinked. This characteristic proves beneficial when attempting to isolate and characterize multi-module platforms. In using generation 5 dendrimers with diameters of approximately 5 nm and over 500 hydrogen bonding sites, the carrying capacity is substantially greater than the previously used dendrons which were approximately 2 nm in diameter and possess 56 hydrogen bonding sites.

Accordingly, the present invention relates to novel therapeutic and diagnostic dendrimer based modular platforms (e.g., drug delivery platforms). In particular, the dendrimer based modular platforms are configured such that two or more dendrimers (e.g., PAMAM dendrimers) are coupled together (e.g., via a cycloaddition reaction) wherein each of the coupled dendrimers is functionalized (e.g., functionalized for targeting, imaging, sensing, and/or providing a therapeutic or diagnostic material and/or monitoring response to therapy). In some embodiments, the present invention provides dendrimer based modular platforms having coupled dendrimers (e.g., two or more coupled dendrimers) wherein each dendrimer is conjugated to one or more functional groups (e.g., therapeutic agent, imaging agent, targeting agent) (e.g., for specific targeting and/or therapeutic use of the dendrimer based modular platform). In some embodiments, the functional groups are conjugated to the dendrimers via covalent attatchment, via a linker, and/or via a triggering agent. In addition, the present invention is directed to methods of synthesizing dendrimer based modular platforms, compositions comprising the dendrimer based modular platforms, as well as systems and methods utilizing the dendrimer based modular platforms (e.g., in diagnostic and/or therapeutic settings (e.g., for the delivery of therapeutics, imaging, and/or targeting agents (e.g., in disease (e.g., cancer) diagnosis and/or therapy, etc.)).

The present invention is not limited to the use of particular types and/or kinds of dendrimers (e.g., a dendrimer conjugated with at least one functional group) (e.g., a dendrimer within a dendrimer based modular platform). Indeed, dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179; herein incorporated by reference in its entirety). In preferred embodiments, the protected core diamine is NH₂—CH₂—CH₂—NHPG. Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process. In some embodiments, the PAMAM dendrimers are “Baker-Huang dendrimers” or “Baker-Huang PAMAM dendrimers” (see, e.g., U.S. Provisional Patent Application Ser. No. 61/251,244; herein incorporated by reference in its entirety).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH; herein incorporated by reference in its entirety). They are water soluble, biocompatible, and cleared from the blood through the kidneys (Peer et al. (2007) Nat. Nanotechnol. 2:751-760; herein incorporated by reference in its entirety) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735; each herein incorporated by reference in its entirety), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462; each herein incorporated by reference in its entirety), and imaging applications (Kobayashi et al. (2003) Bioconj. Chem. 14:388-394; herein incorporated by reference in its entirety).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polyceation non-covalently coupled to the polynucleotide.

Dendrimer-antibody conjugates for use in in vitro diagnostic applications have previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Horn, Science 261:534 (1993)). This allows the controlled degradation of the polymer to release therapeutics at the disease site and provides a mechanism for an external trigger to release the therapeutic agents.

The present invention is not limited to the use of particular therapeutic agents. In some embodiments, the therapeutic agents are effective in treating autoimmune disorders and/or inflammatory disorders (e.g., arthritis). Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen; celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone), TNF-α inhibitors (e.g., adalimumab, certolizumab pegol, etanercept, golimumab, infliximab), EL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agents are effective in treating cancer (see, e.g., U.S. Pat. Nos. 6,471,968, 7,078,461, and U.S. patent application Ser. Nos. 09/940,243, 10/431,682, 11,503,742, 11,661,465, 11/523,509, 12/403,179, 12/106,876, 11/827,637, and 61/101,461; and U.S. Provisional Patent Application Serial Nos. 61/256,759, 61/140,840, 61/091,608, 61/097,780, 61/101,461, 61/237,172, 61/229,168, 61/221,596, and 61/251,244; each herein incorporated by reference in their entireties).

In some embodiments, the therapeutic agent is conjugated to a trigger agent. The present invention is not limited to particular types or kinds of trigger agents.

In some embodiments, sustained release (e.g., slow release over a period of 24-48 hours) of the therapeutic agent is accomplished through conjugating the therapeutic agent (e.g., directly) (e.g., indirectly through one or more additional functional groups) to a trigger agent that slowly degrades in a biological system (e.g., amide linkage, ester linkage, ether linkage). In some embodiments, constitutively active release of the therapeutic agent is accomplished through conjugating the therapeutic agent to a trigger agent that renders the therapeutic agent constitutively active in a biological system (e.g., amide linkage, ether linkage).

In some embodiments, release of the therapeutic agent under specific conditions is accomplished through conjugating the therapeutic agent (e.g., directly) (e.g., indirectly through one or more additional functional groups) to a trigger agent that degrades under such specific conditions (e.g., through activation of a trigger molecule under specific conditions that leads to release of the therapeutic agent). For example, once a conjugate (e.g., a therapeutic agent conjugated with a trigger agent and a targeting agent) arrives at a target site in a subject (e.g., a tumor, or a site of inflammation), components in the target site (e.g., a tumor associated factor, or an inflammatory or pain associated factor) interact with the trigger agent thereby initiating cleavage of the therapeutic agent from the trigger agent. In some embodiments, the trigger agent is configured to degrade (e.g., release the therapeutic agent) upon exposure to a tumor-associated factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides a therapeutic agent conjugated with a trigger agent that is sensitive to (e.g., is cleaved by) hypoxia (e.g., indolequinone). Hypoxia is a feature of several disease states, including cancer, inflammation and rheumatoid arthritis, as well as an indicator of respiratory depression (e.g., resulting from analgesic drugs).

Advances in the chemistry of bioreductive drug activation have led to the design of various hypoxia-selective drug delivery systems in which the pharmacophores of drugs are masked by reductively cleaved groups. In some embodiments, the trigger agent is utilizes a quinone, N-oxide and/or (hetero)aromatic nitro groups. For example, a quinone present, in a conjugate is reduced to phenol under hypoxia conditions, with spontaneous formation of lactone that serves as a driving force for drug release. In some embodiments, a heteroaromatic nitro compound present in a conjugate (e.g., a therapeutic agent conjugated (e.g., directly or indirectly) with a trigger agent) is reduced to either an amine or a hydroxylamine, thereby triggering the spontaneous release of a therapeutic agent. In some embodiments, the trigger agent degrades upon detection of reduced pO2 concentrations (e.g., through use of a redox linker).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M.D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910; each of which are herein incorporated by reference in their entireties). Several such hypoxia activated pro-drugs have been advanced to clinical investigations, and work in relevant oxygen concentrations to prevent cerebral damage. The present invention is not limited to particular hypoxia-activated trigger agents. In some embodiments, the hypoxia-activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see, e.g., Damen, E. W. P., et al., Bioorganic & Medicinal Chemistry, 2002. 10(1): p. 71-77; herein incorporated by reference in its entirety). For example, in such embodiments, the antagonist is only active when released during hypoxia to prevent respiratory failure.

In some embodiments, the trigger agent is sensitive to (e.g., is cleaved by) and/or associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a dendrimer based modular platform provided herein (e.g., that serve as substrates for lysosomal cathepsin B or other proteases expressed (e.g., overexpressed) in tumor cells. In some embodiments, utilization of a 1,6-elimination spacer/linker is utilized (e.g., to permit release of therapeutic drug post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloprotease (MMP). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., a tumor cell)).

In some embodiments, the trigger agent that is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a dendrimer based modular platform). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, therapeutic agent is conjugated (e.g., directly or indirectly) to a targeting agent. The present invention is not limited to any particular targeting agent. In some embodiments, targeting agents are conjugated to the therapeutic agents for delivery of the therapeutic agents to desired body regions (e.g., to the central nervous system (CNS); to a tissue region associated with an inflammatory disorder and/or an autoimmune disorder (e.g., arthritis)). The targeting agents are not limited to targeting specific body regions.

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, dendrimer based modular platforms can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the targeting agent includes but is not limited to an antibody, receptor ligand, hormone, vitamin, and antigen; however: the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFI R, EGFR, estrogen receptor, FGR2, folate receptor, IL-2 receptor, glycoprotein, and VEGFR. In some embodiments, the receptor ligand is folic acid.

Antibodies can be generated to allow for the targeting of antigens or immunogens (e.g., tumor, tissue or pathogen specific antigens) on various biological targets (e.g., pathogens, tumor cells, normal tissue). Such antibodies include but are not limited to polyclonal, monoclonal, chimeric, single chain, Fab fragments, and an Fab expression library.

In some embodiments, the targeting agent is an antibody. In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443; each herein incorporated by reference in their entireties); human carcinoma antigen (See, e.g., U.S. Pat. Nos. 5,693,763; 5,545,530; and 5,808,005; each herein incorporated by reference in their entireties); TP1 and TP3 antigens from osteocarcinoma cells (See, e.g., U.S. Pat. No. 5,855,866; herein incorporated by reference in its entirety); Thomsen-Friedenreich (TF) antigen from adenocarcinoma cells (See, e.g., U.S. Pat. No. 5,110,911; herein incorporated by reference in its entirety); “KC-4 antigen” from human prostrate adenocarcinoma (See, e.g., U.S. Pat. Nos. 4,708,930 and 4,743,543; each herein incorporated by reference in their entireties); a human colorectal cancer antigen (See, e.g., U.S. Pat. No. 4,921,789; herein incorporated by reference in its entirety); CA125 antigen from cystadenocarcinoma (See, e.g., U.S. Pat. No. 4,921,790; herein incorporated by reference in its entirety); DF3 antigen from human breast carcinoma (See, e.g., U.S. Pat. Nos. 4,963,484 and 5,053,489; each herein incorporated by reference in their entireties); a human breast tumor antigen (See, e.g., U.S. Pat. No. 4,939,240: herein incorporated by reference in its entirety); p97 antigen of human melanoma (See, e.g., U.S. Pat. No. 4,918,164: herein incorporated by reference in its entirety); carcinoma or orosomucoid-related antigen (COR^{A})(See, e.g., U.S. Pat. No. 4,914,021; herein incorporated by reference in its entirety); a human pulmonary carcinoma antigen that reacts with human squamous cell lung carcinoma but not with human small cell lung carcinoma (See, e.g., U.S. Pat. No. 4,892,935; herein incorporated by reference in its entirety); T and Tn haptens in glycoproteins of human breast carcinoma (See, e.g., Springer et al., Carbohydr. Res. 178:271-292 (1988); herein incorporated by reference in its entirety), MSA breast carcinoma glycoprotein termed (See, e.g., Tjandra et al., Br. J. Surg. 75:811-817 (1988); herein incorporated by reference in its entirety); MFGM breast carcinoma antigen (See, e.g., Ishida et al., Tumor Biol. 10:12-22 (1989); herein incorporated by reference in its entirety); DU-PAN-2 pancreatic carcinoma antigen (See, e.g., Lan et al., Cancer Res. 45:305-310 (1985); herein incorporated by reference in its entirety); CAl25 ovarian carcinoma antigen (See, e.g., Hanisch et al., Carbohydr. Res. 178:29-47 (1988); herein incorporated by reference in its entirety); YH206 lung carcinoma antigen (See, e.g., Hinoda et al., (1988) Cancer J. 42:653-658 (1988); herein incorporated by reference in its entirety).

In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214; herein incorporated by reference in its entirety). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR)) (see, e.g., Liu, J. K., et al., Neurobiology of Disease, 2005. 19(3): p. 407-418; herein incorporated by reference in its entirety).

In some embodiments, the dendrimer (e.g., a dendrimer conjugated with at least one functional group) (e.g., a dendrimer within a dendrimer based modular platform) is conjugated (e.g., directly or indirectly) to an imaging agent. A multiplicity of imaging agents find use in the present invention. In some embodiments, a dendrimer based modular platform comprises at least one imaging agent that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

In some embodiments, once a component(s) of a targeted dendrimer (e.g., a dendrimer within a dendrimer based modular platform) has attached to (or been internalized into) a target cell (e.g., tumor cell and or inflammatory cell), one or more modules serves to image its location. In some embodiments, chelated paramagnetic ions, such as Gd(III)-diethylenetriaminepentaacetic acid (Gd(III)-DTPA), are conjugated to a dendrimer based modular platform. Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof.

Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

Dendrimer based modular platforms allow functional microscopic imaging of tumors and provide improved methods for imaging. The methods find use in vivo, in vitro, and ex vivo. For example, in one embodiment, dendrimer functional groups are designed to emit light or other detectable signals upon exposure to light. Although the labeled functional groups may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997); herein incorporated by reference in its entirety). In embodiments where the imaging agents are present in deeper tissue, longer wavelengths in the Near-infrared (NMR) are used (See e.g., Lester et al., Cell Mol. Biol. 44:29 (1998); herein incorporated by reference in its entirety). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In some embodiments, the present invention provides dendrimers (e.g., a dendrimer within a dendrimer based modular platform) having a biological monitoring component. The biological monitoring or sensing component of a dendrimer is one that can monitor the particular response in a target cell (e.g., tumor cell), induced by an agent (e.g., a therapeutic agent provided by a dendrimer based modular platform). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In some embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the imaging agent. Fluorescein is easily attached to the dendrimer surface via the isothiocyanate derivatives, available from MOLECULAR PROBES, Inc. This allows the dendrimer based modular platform or components thereof to be imaged with the cells via confocal microscopy. Sensing of the effectiveness of the dendrimer based modular platform or components thereof is preferably achieved by using fluorogenic peptide enzyme substrates. For example, apoptosis caused by the therapeutic agent results in the production of the peptidase caspase-1 (ICE). CALBIOCHEM sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (See, e.g., Talanian et al., J. Biol. Chem., 272: 9677 (1997); herein incorporated by reference in its entirety). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm). In some embodiments, the lysine end of the peptide is linked to pro-drug complex, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (see, e.g., Abrams et al., Development 117:29 (1993); herein incorporated by reference in its entirety) and cis-parinaric acid, sensitive to the lipid peroxidation that accompanies apoptosis (see, e.g., Hockenbery et al., Cell 75:241 (1993); herein incorporated by reference in its entirety). It should be noted that the peptide and the fluorescent dyes are merely exemplary. It is contemplated that any peptide that effectively acts as a substrate for a caspase produced as a result of apoptosis finds use with the present invention.

In some embodiments, conjugation between a dendrimer (e.g., terminal arm of a dendrimer) (e.g., a dendrimer within a dendrimer based modular platform) and a functional group or between functional groups is accomplished through use of a 1,3-dipolar cycloaddition reaction (“click chemistry”). ‘Click chemistry’ involves, for example, the coupling of two different moieties (e.g., a therapeutic agent and a functional group) (e.g., a first functional group and a second functional group) via a 1,3-dipolar cycloaddition reaction between an alkyne moiety (or equivalent thereof) on the surface of the first moeity and an azide moiety (e.g., present on a triazine composition of the present invention) (or equivalent thereof) (or any active end group such as, for example, a primary amine end group, a hydroxyl end group, a carboxylic acid end group, a thiol end group, etc.) on the second moiety. ‘Click’ chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; each herein incorporated by reference in their entireties).

In some embodiments, conjugation between a dendrimer (e.g., a terminal arm of a dendrimer) (e.g., a dendrimer within a dendrimer based modular platform) and a functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., U.S. patent App. No. 61/226,993, herein incorporated by reference in its entirety).

The present invention is not limited by the type of therapeutic agent delivered via dendrimer based modular platforms of the present invention. For example, a therapeutic agent may be any agent selected from the group comprising, but not limited to, autoimmune disorder agent and/or an inflammatory disorder agent. Additional examples of therapeutic agents include, but are not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein.

It is contemplated that components of dendrimer based modular platforms of the present invention provide therapeutic benefits to patients suffering from medical conditions and/or diseases (e.g., cancer, inflammatory disease, chronic pain, autoimmune disease, etc.).

Indeed, in some embodiments of the present invention, methods and compositions are provided for the treatment of inflammatory diseases (e.g., dendrimers conjugated with therapeutic agents configured for treating inflammatory diseases). Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

In some embodiments, the dendrimer based modular platforms configured for treating autoimmune disorders and/or inflammatory disorders (e.g., rheumatoid arthritis) are co-administered to a subject (e.g., a human suffering from an autoimmune disorder and/or an inflammatory disorder) a therapeutic agent configured for treating autoimmune disorders and/or inflammatory disorders (e.g., rheumatoid arthritis). Examples of such agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the medical condition and/or disease is pain (e.g., chronic pain, mild pain, recurring pain, severe pain, etc.). In some embodiments, the dendrimer conjugates (e.g., a dendrimer conjugated with at least one functional group) (e.g., a dendrimer within a dendrimer based modular platform) are configured to deliver pain relief agents to a subject. In some embodiments, the dendrimer conjugates are configured to deliver pain relief agents and pain relief agent antagonists to counter the side effects of pain relief agents. The dendrimer conjugates are not limited to treating a particular type of pain and/or pain resulting from a disease. Examples include, but are not limited to, pain resulting from trauma (e.g., trauma experienced on a battlefield, trauma experienced in an accident (e.g., car accident)). In some embodiments, the dendrimer conjugates of the present invention (e.g., a dendrimer conjugated with at least one functional group) (e.g., a dendrimer within a dendrimer based modular platform) are configured such that they are readily cleared from the subject (e.g., so that there is little to no detectable toxicity at efficacious doses).

In some embodiments, the disease is cancer. The present invention is not limited by the type of cancer treated using the compositions and methods of the present invention. Indeed, a variety of cancer can be treated including, but not limited to, prostate cancer, colon cancer, breast cancer, lung cancer and epithelial cancer. Similarly, the present invention is not limited by the type of inflammatory disease and/or chronic pain treated using the compositions of the present invention. Indeed, a variety of diseases can be treated including, but not limited to, arthritis (e.g., osteoarthritis, rheumatoid arthritis, etc.), inflammatory bowel disease (e.g., colitis, Crohn's disease, etc.), autoimmune disease (e.g., lupus erythematosus, multiple sclerosis, etc.), inflammatory pelvic disease, etc.

In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

The present invention also includes methods involving co-administration of the dendrimer based modular platforms and components thereof described herein with one or more additional active agents. Indeed, it is a further aspect of this invention to provide methods for enhancing prior art therapies and/or pharmaceutical compositions by co-administering dendrimer based modular platforms of this invention. In co-administration procedures, the agents may be administered concurrently or sequentially. In some embodiments, the dendrimer based modular platforms described herein are administered prior to the other active agent(s). The agent or agents to be co-administered depends on the type of condition being treated. For example, when the condition being treated is arthritis, the additional agent can be an agent effective in treating arthritis (e.g., TNF-α inhibitors such as anti-TNF α monoclonal antibodies (such as REMICADE®, CDP-870 and HUMIRA™ (adalimumab) and TNF receptor-immunoglobulin fusion molecules (such as ENBREL®)(entanercept), IL-1 inhibitors, receptor antagonists or soluble IL-1R a (e.g. KINERET™ or ICE inhibitors), nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX® (celecoxib), VIOXX® (rofecoxib), BEXTRA® (valdecoxib) and etoricoxib, (preferably MMP-13 selective inhibitors), NEUROTIN®; pregabalin, sulfasalazine, low dose methotrexate, leflunomide, hydroxychloroquine, d-penicillamine, auranofin or parenteral or oral gold). The additional agents to be co-administered can be any of the well-known agents in the art, including, but not limited to, those that are currently in clinical use. The determination of appropriate type and dosage of radiation treatment is also within the skill in the art or can be determined with relative ease.

In some embodiments, the composition is co-administered with an anti-cancer agent (e.g., Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; CI-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′ deoxyadenosine; Fludarabine-PO₄; mitoxantrone; mitozolomide; Pentostatin; and Tomudex. One particularly preferred class of anticancer agents are taxanes (e.g., paclitaxel and docetaxel). Another important category of anticancer agent is annonaceous acetogenin.

In some embodiments, the composition is co-administered with a pain relief agent. In some embodiments, the pain relief agents include, but are not limited to, analgesic drugs, anxiolytic drugs, anesthetic drugs, antipsychotic drugs, hypnotic drugs, sedative drugs, and muscle relaxant drugs.

In some embodiments, the analgesic drugs include, but are not limited to, non-steroidal anti-inflammatory drugs, COX-2 inhibitors, and opiates. In some embodiments, the non-steroidal anti-inflammatory drugs are selected from the group consisting of Acetylsalicylic acid (Aspirin), Amoxiprin, Benorylate/Benorilate, Choline magnesium salicylate, Diflunisal, Ethenzamide, Faislamine, Methyl salicylate, Magnesium salicylate, Salicyl salicylate, Salicylamide, arylalkanoic acids, Diclofenac, Aceclofenac, Acemethacin, Alclofenac, Bromfenac, Etodolac, Indometacin, Nabumetone, Oxametacin, Proglumetacin, Sulindac, Tolmetin, 2-arylpropionic acids, Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen, Tiaprofenic acid), N-arylanthranilic acids, Mefenamic acid, Flufenamic acid, Meclofenamic acid, Tolfenamic acid, pyrazolidine derivatives, Phenylbutazone, Ampyrone, Azapropazone, Clofezone, Kebuzone, Metamizole, Mofebutazone, Oxyphenbutazone, Phenazone, Sulfinpyrazone, oxicams, Piroxicam, Droxicam, Lornoxicam, Meloxicam, Tenoxicam, sulphonanilides, nimesulide, licofelone, and omega-3 fatty acids. In some embodiments, the COX-2 inhibitors are selected from the group consisting of Celecoxib, Etoricoxib, Lumiracoxib, Parecoxib, Rofecoxib, and Valdecoxib. In some embodiments, the opiate drugs are selected from the group consisting of natural opiates, alkaloids, morphine, codeine; thebaine, semi-synthetic opiates, hydromorphone, hydrocodone, oxycodone, oxymorphone, desomorphine, diacetylmorphine (Heroin), nicomorphine, dipropanoylmorphine, diamorphine, benzylmorphine, Buprenorphine, Nalbuphine, Pentazocine, meperidine, diamorphine, ethylmorphine, fully synthetic opioids, fentanyl, pethidine, Oxycodone, Oxymorphone, methadone, tramadol, Butorphanol, Levorphanol, propoxyphene, endogenous opioid peptides, endorphins, enkephalins, dynorphins, and endomorphins.

In some embodiments, the anxiolytic drugs include, but are not limited to, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze, Triazolam, serotonin 1A agonists, Buspirone (BuSpar), barbituates, amobarbital (Amytal), pentobarbital (Nembutal), secobarbital (Seconal), Phenobarbital, Methohexital, Thiopental, Methylphenobarbital, Metharbital, Barbexaclone), hydroxyzine, cannabidiol, valerian, kava (Kava Kava), chamomile, Kratom, Blue Lotus extracts, Sceletium tortuosum (kanna) and bacopa monniera.

In some embodiments, the anesthetic drugs include, but are not limited to, local anesthetics, procaine, amethocaine, cocaine, lidocaine, prilocaine, bupivacaine, levobupivacaine, ropivacaine, dibucaine, inhaled anesthetics, Desflurane, Enflurane, Halothane, Isoflurane, Nitrous oxide, Sevoflurane, Xenon, intravenous anesthetics, Barbiturates, amobarbital (Amytal), pentobarbital (Nembutal), secobarbital (Seconal), Phenobarbital, Methohexital, Thiopental, Methylphenobarbital, Metharbital, Barbexaclone)), Benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, Etomidate, Ketamine, and Propofol.

In some embodiments, the antipsychOtic drugs include, but are not limited to, butyrophenones, haloperidol, phenothiazines, Chlorpromazine (Thorazine), Fluphenazine (Prolixin), Perphenazine (Trilafon), Prochlorperazine (Compazine), Thioridazine (Mellaril), Trifluoperazine (Stelazine), Mesoridazine, Promazine, Triflupromazine. (Vesprin), Levomepromazine (Nozinan), Promethazine (Phenergan)), thioxanthenes, Chlorprothixene, Flupenthixol (Depixol and Fluanxol), Thiothixene (Navane), Zuclopenthixol (Clopixol & Acuphase)), clozapine, olanzapine, Risperidone (Risperdal), Quetiapine (Seroquel), Ziprasidone (Geodon), Amisulpride (Solian), Paliperidone (Invega), dopamine, bifeprunox, norclozapine (ACP-104), Aripiprazole (Abilify), Tetrabenazine, and Cannabidiol.

In some embodiments, the hypnotic drugs include, but are not limited to, Barbiturates, Opioids, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, nonbenzodiazepines, Zolpidem, Zaleplon, Zopiclone, Eszopiclone, antihistamines, Diphenhydramine, Doxylamine, Hydroxyzine, Promethazine, gamma-hydroxybutyric acid (Xyrem), Glutethimide, Chloral hydrate, Ethchlorvynol, Levomepromazine, Chlormethiazole, Melatonin, and Alcohol.

In some embodiments, the sedative drugs include, but are not limited to, barbituates, amobarbital (Amytal), pentobarbital (Nembutal), secobarbital (Seconal), Phenobarbital, Methohexital, Thiopental, Methylphenobarbital, Metharbital, Barbexaclone), benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, herbal sedatives, ashwagandha, catnip, kava (Piper methysticum), mandrake, marijuana, valerian, solvent sedatives, chloral hydrate (Noctec), diethyl ether (Ether), ethyl alcohol (alcoholic beverage), methyl trichloride (Chloroform), nonbenzodiazepine sedatives, eszopiclone (Lunesta), zaleplon (Sonata), zolpidem (Ambien), zopiclone (Imovane, Zimovane)), clomethiazole (clomethiazole), gamma-hydroxybutyrate (GHB), Thalidomide, ethchlorvynol (Placidyl), glutethimide (Doriden), ketamine (Ketalar, Ketaset), methaqualone (Sopor, Quaalude), methyprylon (Noludar), and ramelteon (Rozerem).

In some embodiments, the muscle relaxant drugs include, but are not limited to, depolarizing muscle relaxants, Succinylcholine, short acting non-depolarizing muscle relaxants, Mivacurium, Rapacuronium, intermediate acting non-depolarizing muscle. relaxants, Atracurium, Cisatracurium, Rocuronium, Vecuronium, long acting non-depolarizing muscle relaxants, Alcuronium, Doxacurium, Gallamine, Metocurine, Pancuronium, Pipecuronium, and d-Tubocurarine.

In some embodiments, the composition is co-administered with a pain relief agent antagonist. In some embodiments, the pain relief agent antagonists include drugs that counter the effect of a pain relief agent (e.g., an anesthetic antagonist, an analgesic antagonist, a mood stabilizer antagonist, a psycholeptic drug antagonist, a psychoanaleptic drug antagonist, a sedative drug antagonist, a muscle relaxant drug antagonist, and a hypnotic drug antagonist). In some embodiments, pain relief agent antagonists include, but are not limited to, a respiratory stimulant, Doxapram, BIMU-8, CX-546, an opiod receptor antagonist, Naloxone, naltrexone, nalorphine, levallorphan, cyprodime, naltrindole, norbinaltorphimine, buprenorphine, a benzodiazepine antagonist, flumazenil, a non-depolarizing muscle relaxant antagonist, and neostigmine.

Where clinical applications are contemplated, in some embodiments of the present invention, the dendrimer based modular platforms are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the dendrimer based modular platforms are used in conjunction with appropriate salts and buffers to render delivery of the compositions in a stable manner to allow for uptake by target cells. Buffers also are employed when the dendrimer based modular platforms are introduced into a patient. Aqueous compositions comprise an effective amount of the dendrimer based modular platforms to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with vectors, cells, or tissues, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The dendrimer based modular platforms may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some embodiments, a therapeutic agent is released from dendrimer based modular platforms within a target cell (e.g., within an endosome). This type of intracellular release (e.g., endosomal disruption of a linker-therapeutic conjugate) is contemplated to provide additional specificity for the compositions and methods of the present invention. The present invention provides dendrimers with multiple (e.g., 100-150) reactive sites for the conjugation of linkers and/or functional groups comprising, but not limited to, therapeutic agents, targeting agents, imaging agents and biological monitoring agents.

The compositions and methods of the present invention are contemplated to be equally effective whether or not the dendrimer based modular platforms of the present invention comprise a fluorescein (e.g. FITC) imaging agent. Thus, each functional group present in a dendrimer composition is able to work independently of the other functional groups. Thus, the present invention provides dendrimer based modular platforms that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the dendrimer based modular platforms in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, dendrimer based modular platforms are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The dendrimer based modular platforms also may be formulated as inhalants for the treatment of lung cancer and such like.

The dendrimer based modular platforms may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications. Most importantly, extensive work has been performed with dendrimers showing no evidence of toxicity when administered intravenously (Roberts et al., J. Biomed. Mater. Res., 30:53 (1996) and Boume et al., J. Magnetic Resonance Imaging, 6:305 (1996)).

In some embodiments, the present invention also provides a kit comprising a composition comprising one or more dendrimer based modular platforms. In some embodiments, the kit comprises a fluorescent agent or bioluminescent agent.

### EXPERIMENTAL

The following examples are provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461, U.S. patent. application Ser. Nos. 09/940,243, 10/431,682, 11,503,742, 11,661,465, 11/523,509, 12/403,179, 12/106,876, 11/827,637, and 61/101,461; and U.S. Provisional Patent Application Ser. Nos. 61/256,759, 61/140,840, 61/091,608, 61/097,780, 61/101,461, 61/237,172, 61/229,168, 61/221,596, and 61/251,244; and International Patent Application No. PCT/US2009/063738; each herein incorporated by reference in their entireties.

### Example 2

### Design, Synthesis and Biological Functionality of Dendrimer-Based Modular Drug Delivery Platform

### Reagents and Materials

Biomedical grade generation 5 PAMAM (poly(amidoamine)) dendrimer was obtained and purified by dialysis, as previously described (see, e.g., Mullen, D. G.; Bioconjug Chem 2008, 19, (9), 1748-52; herein incorporated by reference in its entirety), to remove lower molecular weight impurities including trailing generation dendrimer defect structures.

MeOH (99.8%), acetic anhydride (99.5%), triethylamine (99.5%), dimethyl sulfoxide (99.9%), fluorescein isothiocyanate (98%), dimethylformamide (99.8%), 143-(dimethylamino)-propyl-3-ethylcarbodiimide HCl (EDC) (98%), (99.5%), acetone (ACS reagent grade ≧99.5%), methyl 3-(4-hydroxyphenyl)propanoate (97%), sodium azide (99.99%), 1-bromo-2-chloroethane (98%), ethyl acetate (EtOAc) (99.5%), copper sulfate pentahydrate (99%), sodium ascorbate, 18-crown-6, K₂CO₃, tetrahydrofuran (99.9%), N,N-diisopropylethylamine benzotriazol-1-yl-oxytripyrrolidinophosphonium hexafluorophosphate (98%), D₂O, NaCl, 1 N HCl, 2 M KOH, and volumetric solutions (0.1 M HCl and 0.1 M NaOH) for potentiometric titration were purchased from Sigma Aldrich Co. and used as received. Hexanes (HPLC grade) and 10,000 molecular weight cut-off centrifugal filters (Amicon Ultra) were from Fisher Scientific. 1× phosphate buffer saline (PBS) (Ph=7.4) was purchased from Invitrogen. Sephadex G-25 and Sephadex LH-20 were purchased from GE Lifesciences.

### Nuclear Magnetic Resonance Spectroscopy

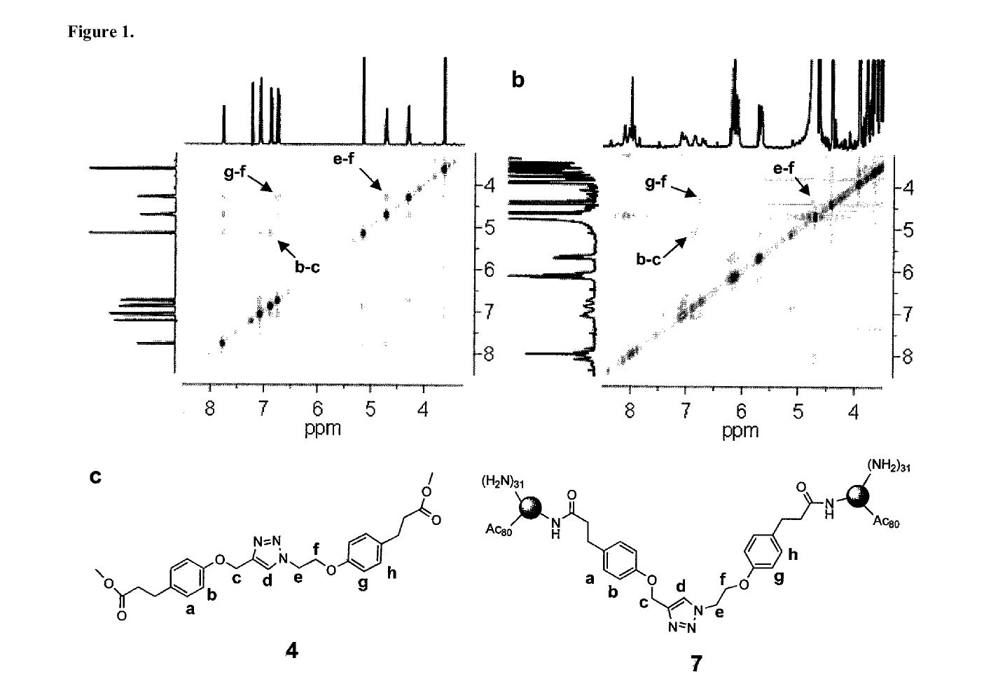

All ¹H NMR experiments were conducted using a Varian Inova 500 MHz instrument. For all dendrimer samples the delay time was 10s. No delay time was used for small molecule samples. NOESY experiments on the dendrimer samples found in FIG. 1 and utilized for Table 1 were also conducted using a Varian Inova 500 MHz instrument. For these experiments the mixing time was 250 ms, the relaxation time was 1s, and the number of increments was 128 with 32 scans per increment. Temperature was controlled at 25° C. The NOESY experiments on the small molecule model system found in FIG. 1 and utilized in Table 1 were conducted using a Varian MR400 (400 MHz) instrument. For the experiments on the small molecules, the mixing time was 800 ms, the relaxation time was 1.2s, and the number of increments was 200 with 4 scans per increment. Based on work published by Hoffman, the internal solvent reference peak for all experiments in CDCl₃ was set to 7.261 ppm. For experiments conducted in D₂O, the internal reference peak was set to 4.717 ppm (see, e.g., Hoffman, R. E., Magn. Reson. Chem. 2006, 44, 606-616; herein incorporated by reference in its entirety).

Table 1: Good correlation is found between the small molecule model system (2a, 3b, and 4) and the dendrimer model system (5, 6, and 7) for the chemical shifts (ppm) of triazole related protons (a-h) both before and after the ‘click’ reaction. Chemical shifts for protons in the model dendrimer system were detected primarily via NOESY experiments.

| Compound | |
|---|---|
| Before Reaction | After Reaction |

| Proton | 2a and 3b | 5 and 6 | 4 | 7 |
|---|---|---|---|---|
| a | 7.14 | 7.13 | 7.11 | 7.09 |
| b | 6.91 | 6.90 | 6.91 | 6.90 |
| c | 4.67 | 4.69 | 5.19 | 5.15 |
| d | n.a. | n.a. | 7.80 | — |
| e | 3.58 | 3.61 | 4.75 | 4.76 |
| f | 4.13 | 4.13 | 4.33 | 4.35 |
| g | 6.85 | 6.90 | 6.78 | 6.74 |
| h | 7.13 | 7.13 | 7.11 | 7.06 |

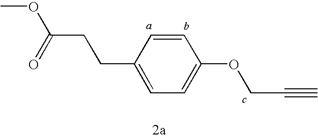

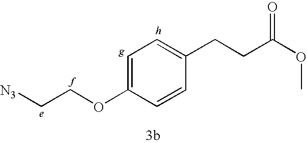

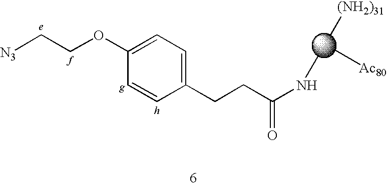

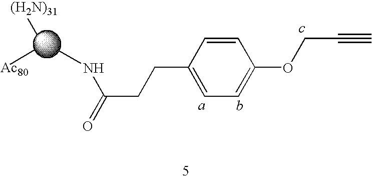

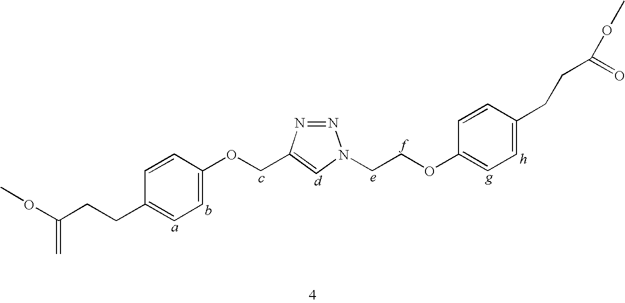

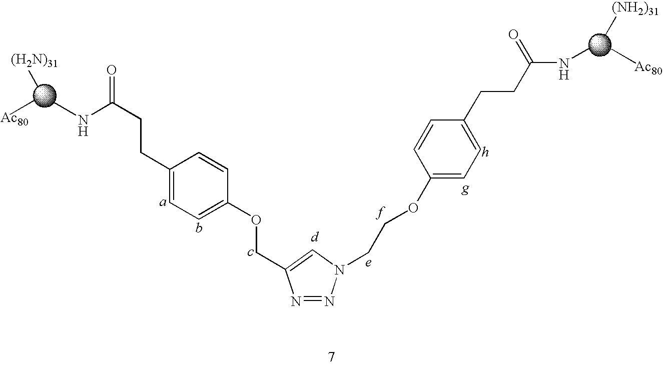

### Gel Permeation Chromatography

GPC experiments were performed on an Alliance Waters 2690 separation module equipped with a 2487 dual wavelength UV absorbance detector (Waters Corporation), a Wyatt Dawn DSP laser photometer, and an Optilab DSP interferometric refractometer (Wyatt Technology Corporation). Columns employed were TosoHaas TSK-Gel Guard PHW 06762 (75 mm×7.5 mm, 12 gm), G 2000 PW 05761 (300 mm×7.5 mm, 10 μm), G 3000 PW 05762 (300 mm×7.5 mm, 10 μm), and G 4000 PW (300 mm×7.5 mm, 17 μm). Column temperature was maintained at 25±0.1° C. with a Waters temperature control module. The isocratic mobile phase was 0.1 M citric acid and 0.025 wt % sodium azide, pH 2.74, at a flow rate of 1 mL/min. The sample concentration was 10 mg/5 mL with an injection volume of 100 μL. The weight average molecular weight, M_{w}, has been determined by GPC, and the number average molecular weight, Mₙ, was calculated with Astra 4.7 software (Wyatt Technology Corporation) based on the molecular weight distribution.

### Reverse Phase High Performance Liquid Chromatography

HPLC analysis was carried out on a Waters Delta 600 HPLC system equipped with a Waters 2996 photodiode array detector, a Waters 717 Plus auto sampler, and Waters Fraction collector III. The instrument was controlled by Empower 2 software. For analysis of the conjugates, a C5 silica-based RP-HPLC column (250×4.6 mm, 300 Å) connected to a C5 guard column (4×3 mm) was used. The mobile phase for elution of the conjugates was a linear gradient beginning with 90:10 (v/v) water/acetonitrile and ending with 10:90 (v/v) water/acetonitrile over 25 min at a flow rate of 1 mL/min. Trifluoroacetic acid (TFA) at 0.14 wt % concentration in water as well as in acetonitrile was used as a counter ion to make the dendrimer surfaces hydrophobic.

### Cell Culture and Treatment

The uptake of the synthesized conjugates was performed using FA-receptor-expressing KB cells (ATCC #CCL-17) as previously described (see, e.g., Thomas et al, J. Med. Chem., 48, 3729-3735, 2005; herein incorporated by reference in its entirety). Cells were maintained in FA-free Roswell Park Memorial Institute-1640 (RPMI 1640) medium supplemented with 10% Fetal Bovine serum (FBS), 2 μM L-glutamine, 100 U/ml penicillin and 100 μg/ml streptomycin in 5% CO₂ at 37° C. Cells were planted into 24 wells plate at density 250,000 per well and allowed to reach ˜90% confluent on the day of the experiment. They were rinsed and incubated in serum free medium with conjugates for 1 hour at 37° C. in 5% CO₂. In some wells, a 20-fold excess of free folic acid or the folic acid conjugated dendrimer was added 30 minutes prior to the addition of the dendrimer platform for the blocking of folate receptors.

### Flow Cytometric Analysis

The cellular fluorescence was quantified on a Beckman-Coulter EPICS-XL MCL flow cytometer, and the data were analyzed using Expo32 software (Beckman-Coulter, Miami, Fla.). Cells were trypsinized, rinsed and suspended in PBS containing 0.1% bovine serum albumin (PBSB). The viable cells were gated, and the mean FL1-fluorescence of 10,000 cells was quantified. Error bars are calculated using the half-peak coefficient of variation (HPCV) (see, e.g., Marie, D.; et al., Biol. Cell 1993, 78, 41-51; herein incorporated by reference in its entirety).

### Synthesis

Dendrimers were identified by the core dendrimer (G5) and conjugated groups:

Ac, Alkyne, Azide, FA, and FITC. In the cases where dendrimers were linked together via the triazole ring, Alkyne and Azide labels are replaced with “L.” Ac refers to the acetamide termination, alkyne to linker 2b, azide to linker 3c, FA to folic acid, and FITC to fluorescein isothiocyanate.

### 1. Synthesis of Partially Acetylated Dendrimer

Partial acetylation of generation 5 PAMAM dendrimer was previously described (see, e.g., Majoros, I. J.; et al., Macromolecules 2003, 36, (15), 5526-5529; Mullen, D. G.; et al., Bioconjug Chem 2008, 19, (9), 1748-52; each herein incorporated by reference in their entireties). The number average molecular weight (27,336 g/mol) and PDI (1.018) of the un-acetylated dendrimer was determined by GPC. Potentiometric titration was conducted to determine the average number of primary amines per dendrimer (112). Three batches of partially acetylated dendrimer were synthesized for further modification. 1′ G5Ac_{72%}: ¹H NMR integration determined the degree of acetylation to be 72%. Number average molecular weight (30,722 g/mol) was computed based on the addition of mass to the dendrimer from the acetyl groups as determined by NMR. PDI (1.019) of the purified acetylated dendrimer were determined by GPC. 1″ G5Ac_{65%}: ¹H NMR integration determined the degree of acetylation to be 65%. Number average molecular weight (30,394 g/mol) was computed based on the addition of mass to the dendrimer from the acetyl groups as determined by NMR. PDI (1.060) of the purified acetylated dendrimer were determined by GPC. 1′″ G5Ac_{67%}: ¹H NMR integration determined the degree of acetylation to be 67%. Number average molecular weight (30,473 g/mol) was computed based on the addition of mass to the dendrimer from the acetyl groups as determined by NMR.

### 2. Synthesis of Alkyne Linker (3-(4-(prop-2-ynyloxy)phenyl)propanoic acid)

2a. Synthesis of methyl 3-(4-(prop-2-ynyloxy)phenyl)propanoate has been previously reported (see, e.g., Mullen, D. G.; et al., Bioconjug Chem 2008, 19, (9), 1748-52; herein incorporated by reference in its entirety).

2b. Synthesis of (3-(4-(prop-2-ynyloxy)phenyl)propanoic acid) has also been previously reported(see, e.g., Mullen, D. G.; et al., Bioconjug Chem 2008, 19, (9), 1748-52; herein incorporated by reference in its entirety).

### 3. Synthesis of Azide Linker (3-(4-(2-azidoethoxy)phenyl)propanoic acid)

3a. To a solution of methyl 3-(4-hydroxyphenyl)propanoate (1.699 g, 9.43 mmole) in dry acetone (47.5 mL) was added anhydrous K₂CO₃ (3.909 g, 0.0283 mole) followed by 1-bromo-2-chloroethane (1.563 mL, 0.01886 mole). The resulting suspension was refluxed for 43 h with vigorous stirring. The reaction mixture was cooled to room temperature and the salt was removed by filtration followed by washing with portions of EtOAc (3×70 mL). The crude material was purified by silica chromatography (25:75 EtOAc:Hexane) and the solvent was removed under vacuum to give the desired product, methyl 3-(4-(2-chloroethoxy)phenyl)propanoate 3a, as an oil (0.75 g, 33%). ¹H NMR (500 MHz, CDCl₃) δ 7.121 (d, J=8.7, 2H), 6.843 (d, J=8.7, 2H), 4.206 (t, J=5.9, 2H), 3.798 (t, J=5.9, 2H), 3.664 (s, 3H), 2.895 (t, J=7.8, 2H), 2.598 (t, J=7.8, 2H).

3b. To a solution of methyl 3-(4-(2-chloroethoxy)phenyl)propanoate 3a (0.75 g 3.1 mmole) in anhydrous DMF (6.1 mL) was added 18-crown-6 (3.4 mg, 0.013 mmole) and sodium azide (0.44 g, 6.8 mmole). The resulting solution was heated at 78° C. for 11 h. The reaction mixture was cooled to room temperature, diluted with ethyl acetate (50 mL), washed with a saturated NaHCO₃ solution (4×70 mL), and then dried over MgSO₄. The solvent was removed under vacuum to give methyl 3-(4-(2-azidoethoxy)phenyl)propanoate 3b as a yellow oil (0.58 g, 75%) ¹H NMR (500 MHz, CDCl₃) δ 7.125 (d, J=8.6, 2H), 6.849 (d, J=8.6, 2H), 4.129 (t, J=5.0 2H), 3.666 (s, 3H), 3.581 (t, J=5.0, 2H), 2.899 (t, J=7.8, 2H), 2.600 (t, J=7.8, 2H).

3c. To a solution of methyl 3-(4-(2-azidoethoxy)phenyl)propanoate 3b (3.88 g, 0.0156 mole) in methanol (102 mL) was added potassium hydroxide (2 M, 28.3 mL, 0.0566 mole). The resulting solution was refluxed at 70° C. for 3 h. The solution was cooled to room temperature and condensed under reduced pressure. The residue was dissolved in water (30 mL) and was acidified by addition of 1 N HCl to pH 1. The white cloudy solution was diluted with EtOAc. Layers were separated and the aqueous layer was extracted with EtOAc (2×70 mL). The combined organic extracts were washed with a saturated NaCl solution and dried over MgSO₄. Solvent was evaporated under vacuum to give the (3-(4-(2-azidoethoxy)phenyl)propanoic acid) 3c as a white solid (3.44 g, 93.9%). ¹H NMR (500 MHz, CDCl₃) δ 7.139 (d, J=8.5, 2H), 6.859 (d, J=8.5, 2H), 4.132 (t, J=5.0 2H), 3.584 (t, J=5.0, 2H), 2.909 (t, J=7.7, 2H), 2.653 (t, J=7.7, 2H).

### 4. Synthesis of Small Molecule Model System

The methyl-ester forms of the Alkyne Linker 2a (448.0 mg, 1.80 mmole) and Azide Linker 3b (371.5 mg, 1.70 mmole) were dissolved in a mixture of THF (1.6 mL) and water (0.4 mL). To this mixture was added copper sulfate pentahydrate (43.1 mg, 86.0 μmole) and sodium ascorbate (170.9 mg, 431 μmole). The resulting reaction mixture was stirred at room temperature for 24 hrs. The solution was then diluted in EtOAc (60 mL) and water (60 mL). Layers were separated and the aqueous layer was extracted twice with EtOAc solution (2×70 mL). The combined organic extracts were washed with a saturated NaHCO₃ solution (3×70 mL) and then with saturated NaCl solution (2×70 mL). The organic extracts were finally dried over MgSO₄. Solvent was evaporated under reduced pressure to give the desired product 4 as a white solid (0.54 g, 95%). NMR (500 MHz, CDCl₃) δ 7.799 (s, 1H), 7.108 (overlapping d, J=8.4, 4H), 6.911 (d, J=8.6, 2H), 6.778 (d, J=8.6, 2H), 5.185 (s, 2H), 4.749 (t, J=5.0, 2H), 4.329 (t, J=5.0, 2H), 3.663 (s, 3H), 3.657 (s, 3H), 2.889 (t, J=7.8, 2H), 2.885 (t, J=7.7, 2H), 2.592 (t, J=7.8, 2H), 2.586 (t, J=7.7, 2H).

### 5. Synthesis of G5-Ac_{72%}-Alkyne₁.

A solution of partially acetylated dendrimer (54.6 mg, 1.78 μmmole) was prepared with anhydrous DMSO (12.133 mL). The Alkyne Linker 2b (0.9 mg, 4.4 μmole) was dissolved in DMSO (453 μL) and add to the dendrimer-DMSO solution. To this mixture was added N,N-Diisopropylethylamine (4.6 μL, 26 μmole) and the resulting solution was stirred for 45 minutes. Benzotriazol-1-yl-oxytripyrrolidinophosphonium hexafluorophosphate (2.3 mg, 4.4 μmole) was dissolved in DMSO (462 μL) and added in a dropwise manner to the dendrimer/Alkyne Linker solution. The resulting solution was stirred for 24 hrs. All reaction steps were carried out in glass flasks at room temperature under nitrogen.

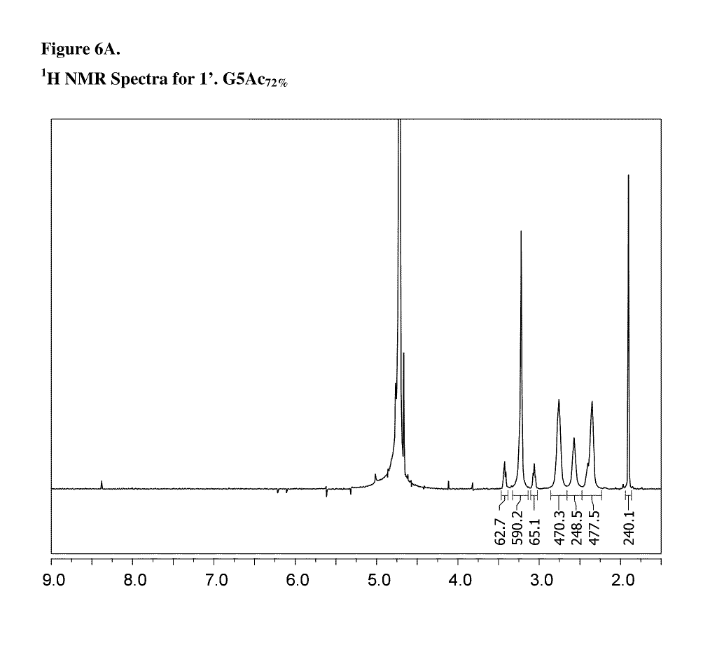

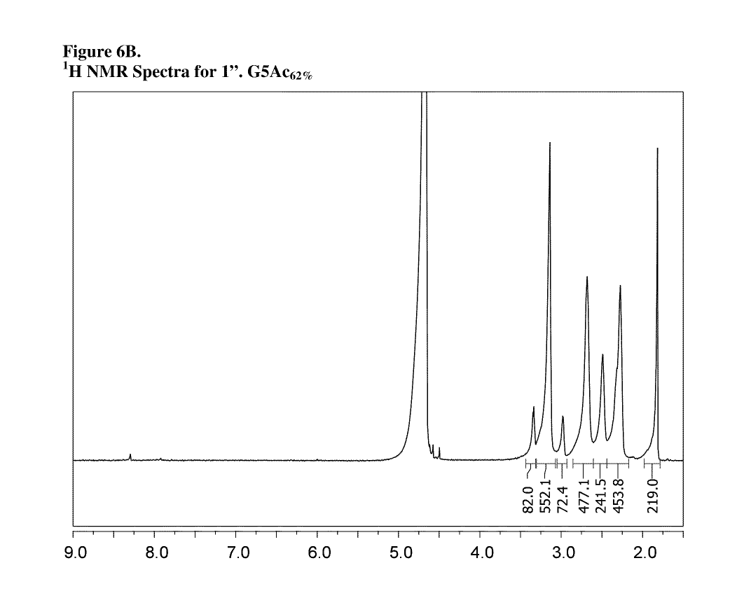

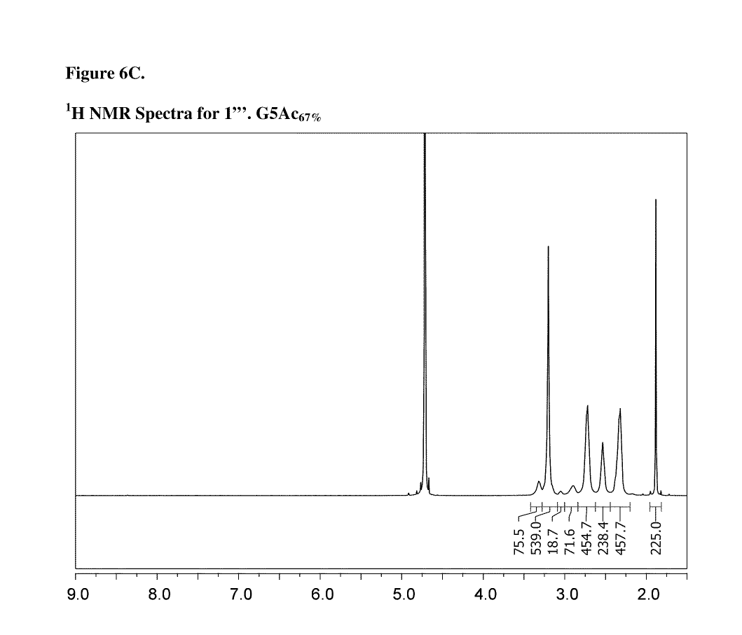

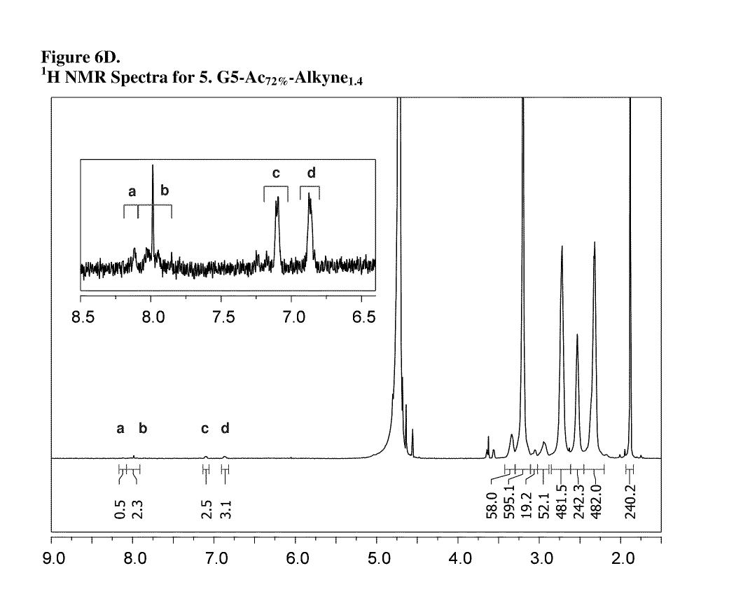

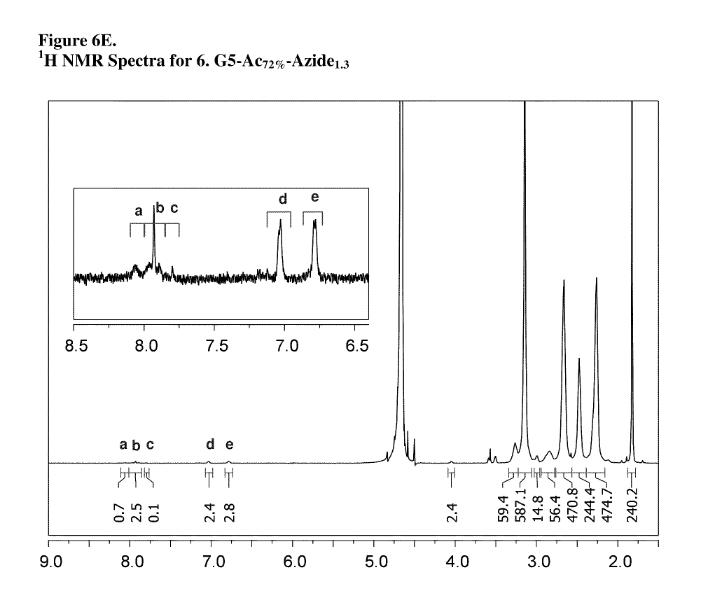

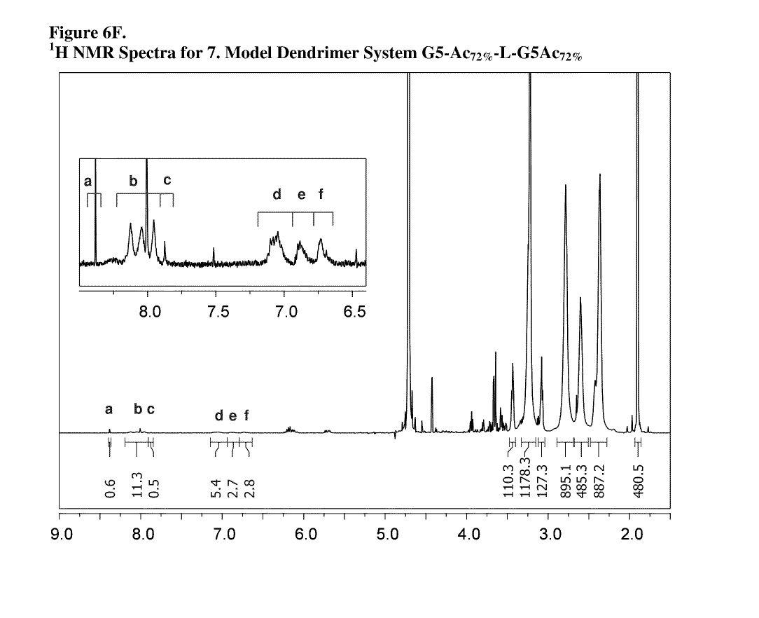

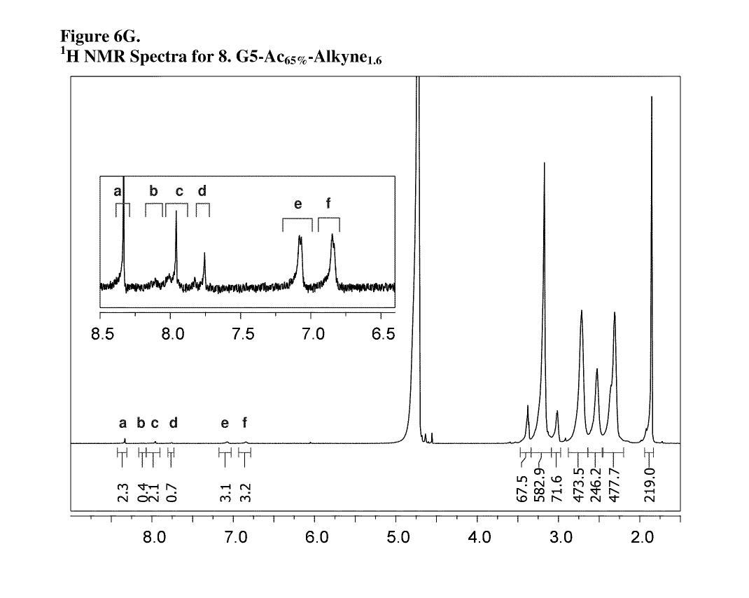

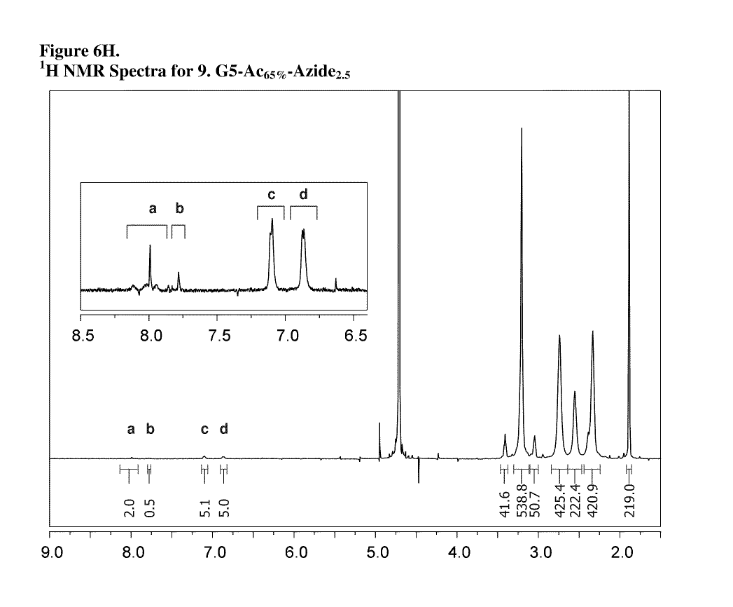

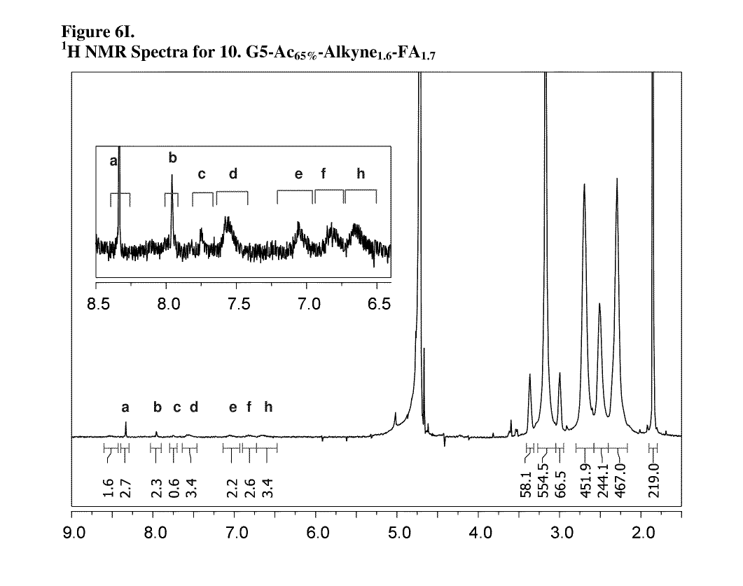

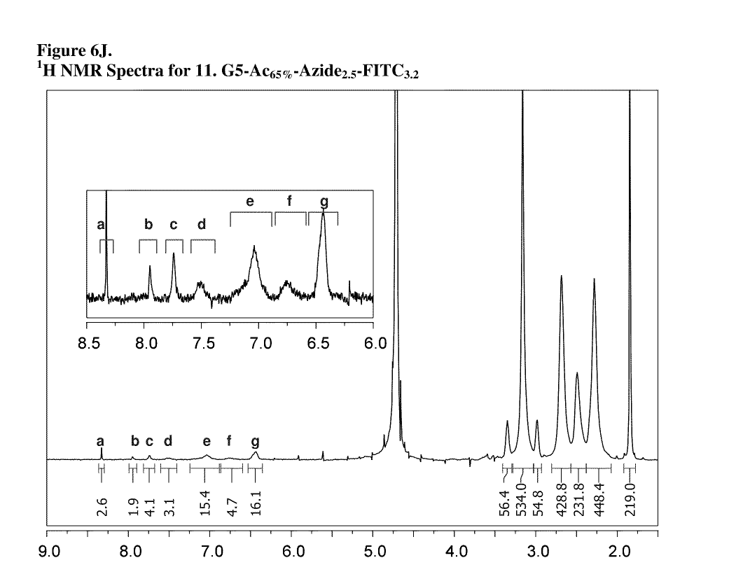

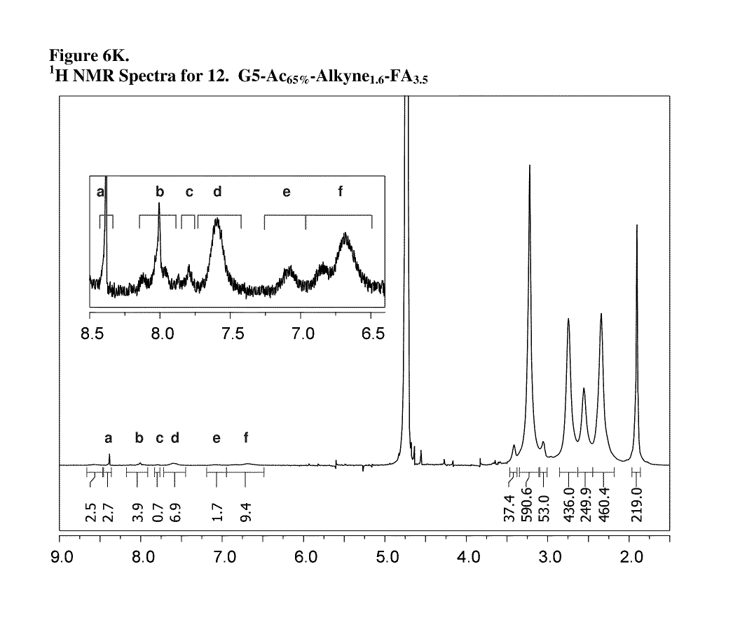

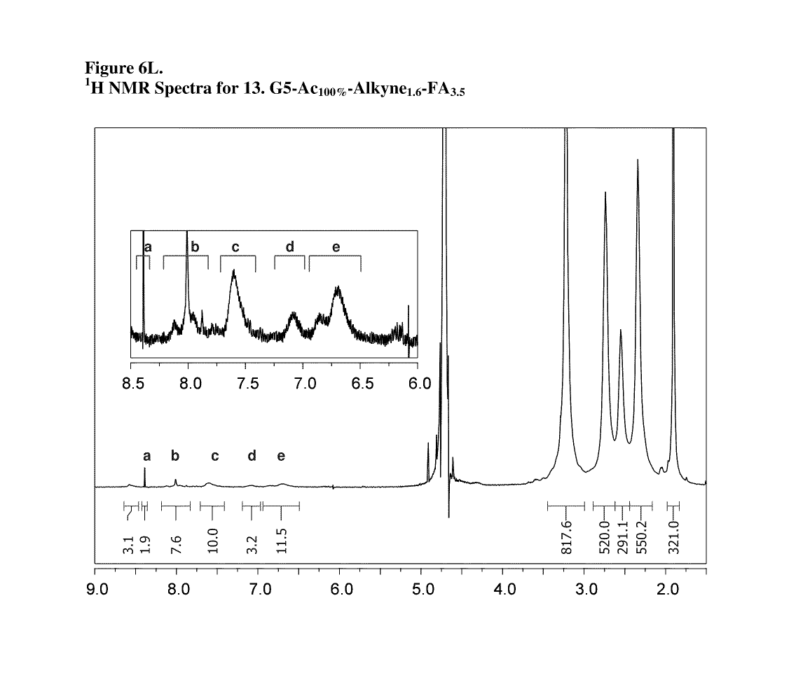

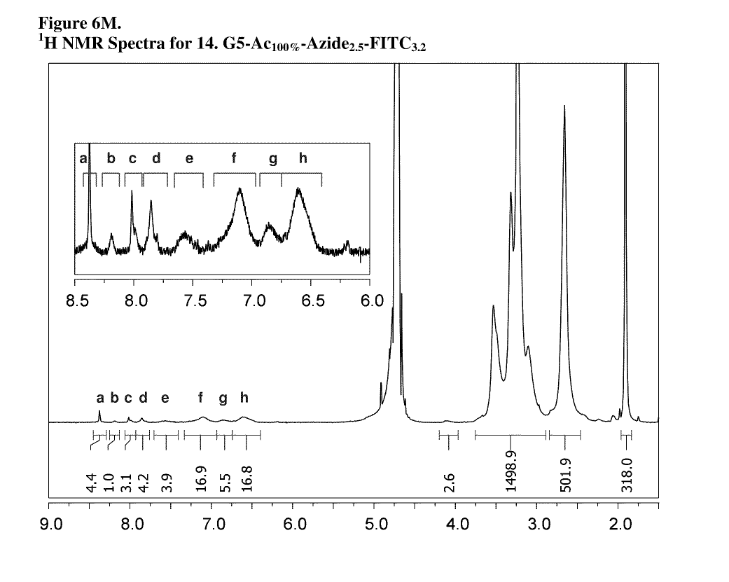

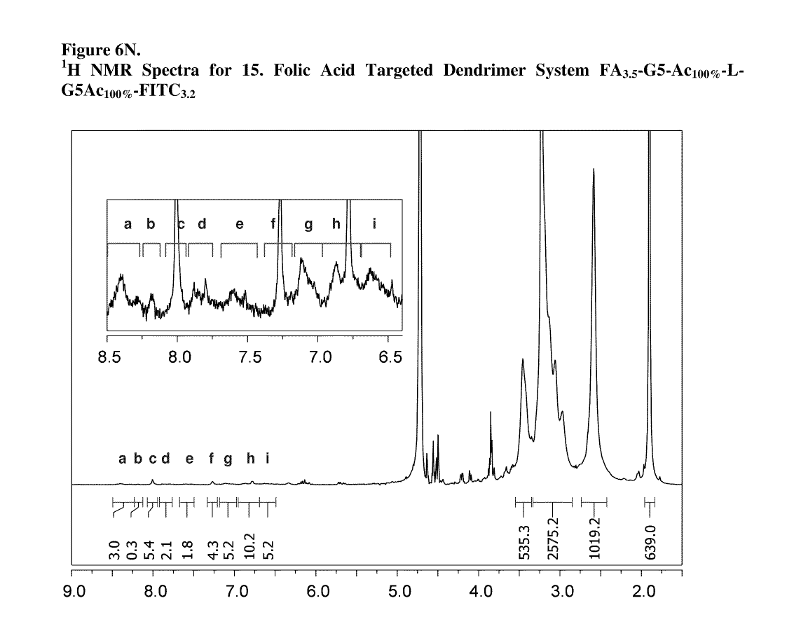

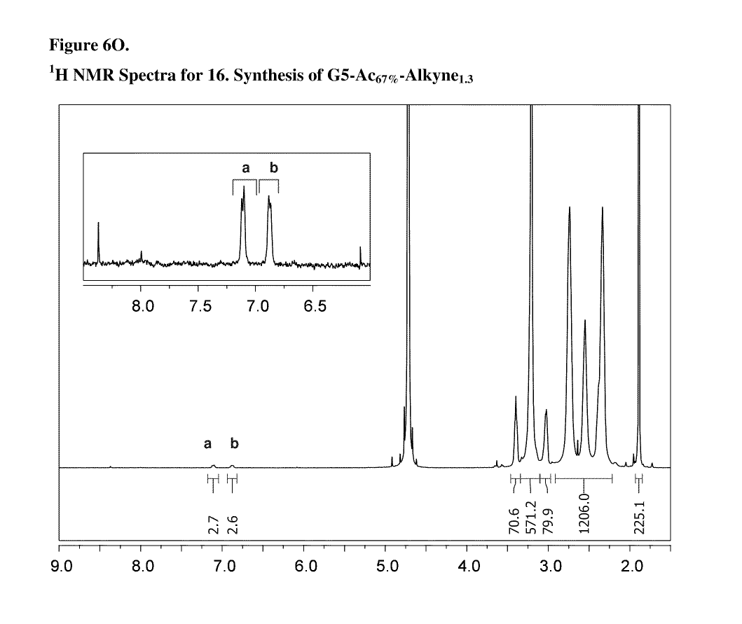

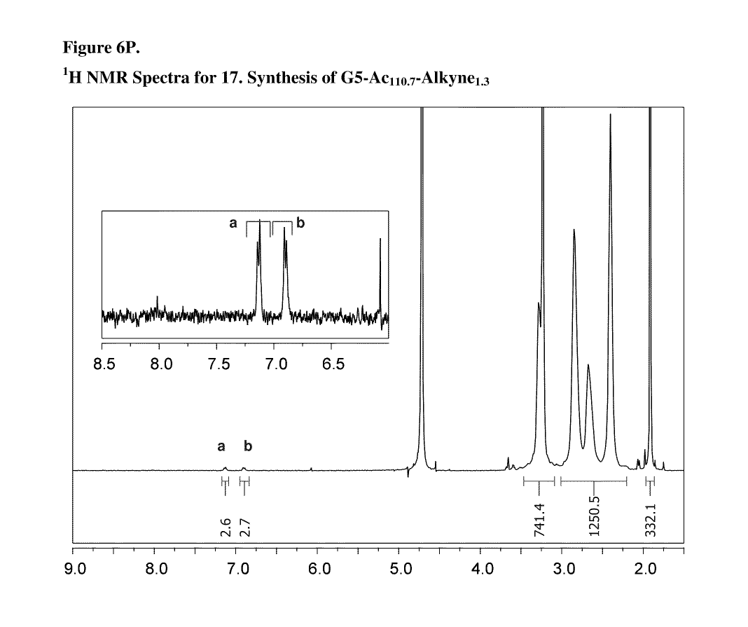

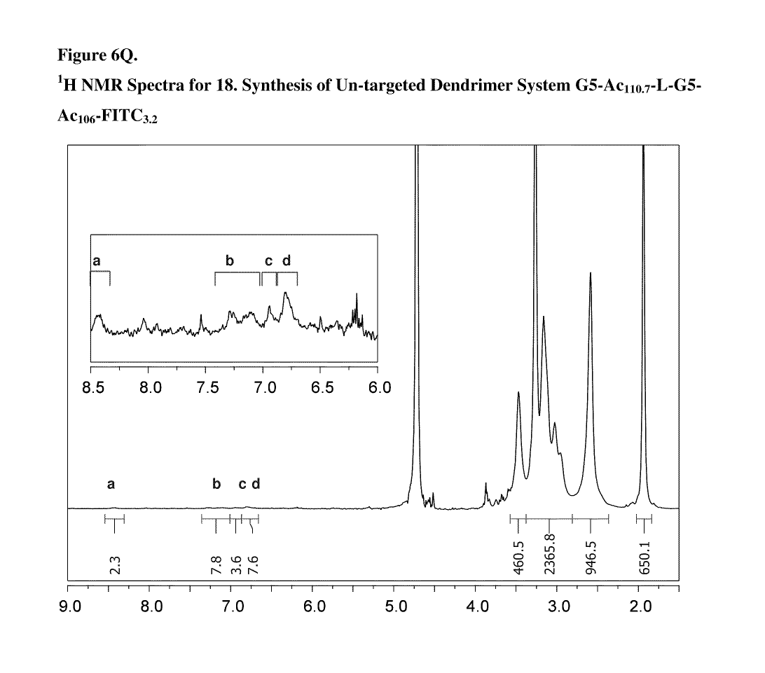

The reaction mixture was purified using 10,000 MWCO centrifugal filtration devices. Purification consisted of two cycles using 1×PBS and eight cycles using DI water. All cycles were 10 minutes at 5,000 rpm. The resulting product 5 was lyophilized for three days to yield a white solid (41.5 mg, 75.4%). ¹H NMR integration determined an average of 1.4 Alkyne Linkers coupled to the dendrimer. The quantification of the number of linkers by NMR integration is described previously (see, e.g., Mullen, D. G.; et al., Bioconjug. Chem. 2008, 19, 1748-52; herein incorporated by reference in its entirety). ¹H NMR spectrum for the resulting product 5 is shown in FIG. 6D.

### 6. Synthesis of G5-Ac_{72%}-Azide_{1.3}

A solution of partially acetylated dendrimer (60.5 mg, 1.97 μmole) was prepared with anhydrous DMSO (13.444 mL). The Azide Linker 3c (1.2 mg, 4.9 μmole) was dissolved in DMSO (578 μL) and add to the dendrimer-DMSO solution. To this mixture was added N,N-Diisopropylethylamine (5.1 μL, 30 μmole) and the resulting solution was stirred for 45 minutes. Benzotriazol-1-yl-oxytripyrrolidinophosphonium hexafluorophosphate (2.6 mg, 4.9 μmole) was dissolved in DMSO (511 μL) and added in a dropwise manner to the dendrimer/Azide Linker solution. The resulting solution was stirred for 24 hrs. All reaction steps were carried out in glass flasks at room temperature under nitrogen.

The reaction mixture was purified using 10,000 MWCO centrifugal filtration devices. Purification consisted of two cycles using 1×PBS and eight cycles using DI water. All cycles were 10 minutes at 5,000 rpm. The resulting product 6 was lyophilized for three days to yield a white solid (50.3 mg, 91.3%). ¹H NMR integration determined an average of 1.3 Alkyne Linkers coupled to the dendrimer. ¹H NMR spectrum for the resulting product 6 is shown in FIG. 6E.

### 7. Synthesis of Model Dendrimer System G5-Ac_{72%}-L-G5Ac_{72%}

Partially acetylated dendrimer with an average of 1.4 Alkyne Linkers 5 (15.30 mg, 0.493 μmole) and partially acetylated dendrimer with an average of 1.3 Azide Linkers 6 (15.4 mg, 0.496 μmole) was dissolved in deuterium oxide (0.820 mL) and placed in a glass microwave reactor vessel. Sodium ascorbate (6.9 mg, 35 μmole) and copper sulfate pentahydrate (5.9 mg, 24 μmole) was added to the dendrimer solution. The resulting solution was placed in a microwave reactor for 40 seconds at 100 watts with a cut-off temperature of 100° C. The microwave conditions were repeated for an additional 40 seconds. The cut-off temperature was then increased to 110° C. and the microwave was run at 100 watts for 2 minutes. The resulting crude product was a turbid yellow. The crude product was transferred to an NMR tube and analyzed by NOESY and ¹H NMR. After two days, the crude product in solution turned to a red-brown solution with a precipitate at the bottom of the NMR tube. NOESY and ¹H NMR experiments were repeated at this time point. The supernatant was separated from the precipitate and lyophilized to yield 4.9 mg of a brown solid. ¹H NMR spectrum for the resulting product 7 is shown in FIG. 6F.

### 8. Synthesis of G5-Ac_{65%}-Alkyne_{1.6}

The Alkyne Linker was conjugated to the partially acetylated dendrimer in two consecutive reactions. First, a stock solution of the Alkyne Linker 2b (9.5 mg, 0.047 mmole) was generated with a mixture of DMF (6.198 mL) and DMSO (3.099 mL). To this mixture was added EDC (124.9 mg, 0.651 mmole). The resulting solution was stirred for 2 h at room temperature to create the active ester form of the Alkyne Linker.

A solution of partially acetylated dendrimer 1″ (58.8 mg, 1.930 mmole) was prepared with DI water (13.099 mL). The active ester form of the Alkyne Linker (5.784 mL, 0.0289 mmole) in DMF/DMSO was added in a dropwise manner (0.13 mL/min) to the dendrimer-water solution. The resulting reaction mixture was stirred for 2 days.All reaction steps were carried out in glass flasks at room temperature under nitrogen. The reaction mixture was purified using 10,000 MWCO centrifugal filtration devices. Purification consisted of five cycles using 1×PBS and five cycles using DI water. All cycles were 30 minutes at 5,000 rpm. The resulting product 8 was lyophilized for three days to yield a white solid (55.0 mg, 92.5%). ¹H NMR integration determined an average of 1.6 Alkyne Linkers coupled to the dendrimer. ¹H NMR spectrum for the resulting product 8 is shown in FIG. 6G.

### 9. Synthesis of G5-Ac_{65%}-Azide_{2.5}

The Azide Linker was conjugated to the partially acetylated dendrimer in two consecutive reactions. First, a stock solution of the Azide Linker 3c (7.6 mg, 0.032 mmole) was generated with a mixture of DMF (4.958 mL) and DMSO (2.479 mL). To this mixture was added EDC (86.7 mg, 0.452 mmole). The resulting solution was stirred for 1.75 h at room temperature to create the active ester form of the Azide Linker.

A solution of partially acetylated dendrimer 1″ (58.8 mg, 1.930 μmole) was prepared with DI water (13.099 mL). The active ester form of the Azide Linker (6.663 mL, 0.0289 mmole) in DMF/DMSO was added in a dropwise manner (0.13 mL/min) to the first dendrimer-water aliquot. The resulting mixture was stirred for 2 days. All reaction steps were carried out in glass flasks at room temperature under nitrogen. The reaction mixture was purified using 10,000 MWCO centrifugal filtration devices. Purification consisted of five cycles using 1×PBS and five cycles using DI water. All cycles were 10 minutes at 5,000 rpm. The resulting product 9 was lyophilized for three days to yield a white solid (55.0 mg, 88.8%). ¹H NMR integration determined an average of 2.5 Azide linkers coupled to the dendrimer. ¹H NMR spectrum for the resulting product 9 is shown in FIG. 6H.

### 10. Synthesis of G5-Ac_{65%}-Alkyne_{1.6}-FA_{1.7}

Folic acid was conjugated to the Alkyne Linker-conjugated dendrimer 8 in two consecutive reactions. First, a solution of folic acid (1.9 mg, 4.26 μmole) was generated with a mixture of DMF (1.234 mL) and DMSO (0.617 mL). To this mixture was added EDC (11.4 mg, 59.7 μmmole). The resulting solution was stirred for 1 h at room temperature to create the active ester form of folic acid.

A solution of partially acetylated dendrimer with an average number of 1.6 Alkyne Linkers 8 (20.8 mg, 0.752 μmole) was prepared with DI water (4.638 mL). The active ester form of folic acid (1.850 mL, 4.26 μmole) in DMF/DMSO was added in a dropwise manner to the dendrimer-water solution. The resulting reaction mixture was stirred for 3 days. All reaction steps were carried out in glass flasks at room temperature under nitrogen. The reaction mixture was purified using 10,000 MWCO centrifugal filtration devices. Purification consisted of five cycles using 1×PBS and four cycles using DI water. All cycles were 10 minutes at 5,000 rpm. The resulting product 10 was lyophilized for three days to yield a white solid (15.6 mg, 73.2%). ¹H NMR integration determined an average of 1.7 folic acid molecules coupled to the dendrimer. ¹H NMR spectrum for the resulting product 10 is shown in FIG. 6I.

### 11. Synthesis of G5-Ac_{65%}-Azide_{2.5}-FITC_{3.2}

Partially acetylated dendrimer with an average number of 2.5 Azide Linkers 9 (21.5 mg, 0.694 μmole) was dissolved in DMSO (1.5 mL). Fluorescene isothiocyanate (1.4 mg, 3.5 μmole) was dissolved in DMSO (0.54 mL) and added in a dropwise fashion to the dendrimer solution. The resulting mixture was stirred for 24 hours at room temperature. The reaction mixture was purified using 10,000 MWCO centrifugal filtration devices. Purification consisted of six cycles using 1×PBS and six cycles using DI water. All 1×PBS cycles were 15 minutes at 5,000 rpm and all DI water cycles were 15 minutes at 5,000 rpm. HPLC analysis of the lyophilized product detected un-conjugated FITC remaining in the sample. To remove the remaining un-reacted FITC, the conjugate was purified by size exclusion chromatography using Sephadex G-25 beads in 1×PBS. The dendrimer fraction was collected and the elution buffer was exchanged with DI water using 10,000 MWCO centrifugal filtration devices (four cycles of 10 minutes at 5,000 rpm). The purified product 11 was lyophilized to yield a yellow-orange solid (10.1 mg, 45.6%). ¹H NMR integration determined an average of 3.2 FITC coupled to the dendrimer. NMR spectrum for the purified product 11 is shown in FIG. 6J.

### 12. Synthesis of G5-Ac_{65%}-Alkyne_{1.6}-FA_{3.5}

Additional folic acid was conjugated to the partially acetylated dendrimer with an average of 1.6 Alkyne Linkers and 1.7 folic acid molecules 10 in two consecutive reactions. First, a solution of folic acid (1.1 mg, 2.4 mole) was generated with a mixture of DMF (0.687 mL) and DMSO (0.344 mL). To this mixture was added EDC (6.3 mg, 33 mole). The resulting solution was stirred for 1 h at room temperature to create the active ester form of the folic acid.

A solution of partially acetylated dendrimer with an average number of 1.6 Alkyne Linkers and 1.7 folic acid molecules 10 (8.5 mg, 0.298 μmole) was prepared with DI water (1.895 mL). The active ester form of folic acid (1.031 mL, 2.4 mole) in DMF/DMSO was added in a dropwise manner to the dendrimer-water solution. The resulting reaction mixture was stirred for 3 days. All reaction steps were carried out in glass flasks at room temperature under nitrogen. The reaction mixture was purified by size exclusion chromatography using Sephadex G-25 in 1×PBS. The dendrimer fraction was collected and the elution buffer was exchanged with DI water using 10,000 MWCO centrifugal filtration devices (four cycles of 10 minutes at 5,000 rpm). The purified product 12 was lyophilized for three days to yield a yellow solid (7.0 mg, 80.5%). NMR integration determined an average of 3.5 folic acid molecules coupled to the dendrimer. ¹H NMR spectrum for the purified dendrimer 12 is shown in FIG. 6K.

### 13. Synthesis of G5-Ac₁₀₇-Alkyne_{1.6}-FA_{3.5}

Partially acetylated dendrimer with an average number of 1.6 Alkyne Linkers and 3.5 folic acid 12 (7.0 mg, 0.22 μmole) was dissolved in anhydrous methanol (1.124 mL). Triethylamine (1.7 μL, 0.012 mmole) was added to this mixture and stirred for 30 minutes. Acetic anhydride (0.9 μL, 9.6 μmole) was added in a dropwise manner to the dendrimer solution. The reaction was carried out in a glass flask, under nitrogen, at room temperature for 24 hours. Methanol was evaporated from the resulting solution and the product was purified by size exclusion chromatography using Sephadex LH-20 in methanol. The purified dendrimer 13 was lyophilized for three days to yield a white solid (6.6 mg, 90.3%). ¹H NMR integration determined the degree of acetylation to be 100%. ¹H NMR spectrum for the purified dendrimer 13 is shown in FIG. 6L.

### 14. Synthesis of G5-Ac₁₀₆-Azide_{2.5}-FITC_{3.2}

Partially acetylated dendrimer with an average number of 2.5 Azide Linkers and 3.2 FITC 11 (7.5 mg, 0.23 μmole) was dissolved in anhydrous methanol (1.206 mL). Triethylamine (1.8 μL, 0.013 mmole) was added to this mixture and stirred for 30 minutes. Acetic anhydride (1.0 μL, 10.0 μmole) was added in a dropwise manner to the dendrimer solution. The reaction was carried out in a glass flask, under nitrogen, at room temperature for 24 hours. Methanol was evaporated from the resulting solution and the product was purified by size exclusion chromatography using Sephadex LH-20 in methanol. The purified dendrimer 14 was lyophilized for three days to yield a white solid (7.1 mg, 90.6%). ¹H NMR integration determined the degree of acetylation to be 100%. ¹H NMR spectrum for the purified dendrimer 14 is shown in FIG. 6M.

### 15. Synthesis of Folic Acid Targeted Dendrimer System FA_{3.5}-G5-Ac₁₀₇-L-G5-Ac₁₀₆-FITC_{3.2}

Dendrimer with an average of 1.6 Alkyne Linkers and 3.5 folic acid molecules 13 (3.1 mg, 91 nmole) and dendrimer with an average of 2.5 Azide Linkers and 3.2 FITC molecules 14 (3.0 mg, 88 nmole) were dissolved in deuterium oxide (0.650 mL) and placed in a glass microwave reactor vessel. Sodium ascorbate (1.1 mg, 4.5 μmole) and copper sulfate pentahydrate (1.1 mg, 5.4 μmole) was added to the dendrimer solution. The resulting solution was placed in a microwave reactor for 6.5 minutes at 100 watts with a cut-off temperature of 100° C. The reaction mixture was transferred to an NMR tube and analyzed by NOESY and ¹H NMR spectroscopy using. Lyophilization yielded 6.7 mg of a red solid. ¹H NMR spectrum for 15 is shown in FIG. 6N.

### 16. Synthesis of G5-Ac_{67%}-Alkyne_{1.3}

A solution of partially acetylated dendrimer 1′″ (176.7 mg, 5.8 μmole) was prepared with anhydrous DMSO (39.27 mL). The Alkyne Linker 2b (2.6 mg, 13 μmole) was dissolved in DMSO (1.306 mL) and add to the dendrimer-DMSO solution. To this mixture was added N,N-Diisopropylethylamine (13.4 μL, 76.7 μmole) and the resulting solution was stirred for 45 minutes. Benzotriazol-1-yl-oxytripyrrolidinophosphonium hexafluorophosphate (6.7 mg, 13 μmole) was dissolved in DMSO (1.331 mL) and added in a dropwise manner to the dendrimer/Alkyne Linker solution. The resulting solution was stirred for 24 hrs. All reaction steps were carried out in glass flasks at room temperature under nitrogen.

The reaction mixture was purified using 10,000 MWCO centrifugal filtration devices. Purification consisted of two cycles using 1×PBS and eight cycles using DI water. All cycles were 10 minutes at 5,000 rpm. The resulting product 5 was lyophilized for three days to yield a white solid (116.2 mg, 65.2%). ¹H NMR integration determined an average of 1.3 Alkyne Linkers coupled to the dendrimer. ¹H NMR spectrum for 16 is shown in FIG. 6O.

### 17. Synthesis of G5-Ac_{110.7}-Alkyne_{1.3}

Partially acetylated dendrimer with an average number of 1.3 Alkyne Linkers 16 (22.6 mg, 0.737 μmole) was dissolved in anhydrous methanol (3.0 mL). Triethylamine (5.8 μL, 0.042 mmole) was added to this mixture and stirred for 30 minutes. Acetic anhydride (3.2 μL, 34 μmmole) was added in a dropwise manner to the dendrimer solution. The reaction was carried out in a glass flask, under nitrogen, at room temperature for 24 hours. Methanol was evaporated from the resulting solution and the product was purified by size exclusion chromatography using Sephadex LH-20 in methanol. The purified dendrimer 17 was lyophilized for three days to yield a white solid (19.1 mg, 80.5%). ¹H NMR integration determined the degree of acetylation to be 100%. ¹H NMR spectrum for 17 is shown in FIG. 6P.

### 18. Synthesis of Un-targeted Dendrimer System G5-Ac_{110.7}-L-G5-Ac₁₀₆-FITC_{3.2}

Dendrimer with an average of 1.3 Alkyne Linkers 17 (1.0 mg, 31 nmole) and dendrimer with an average of 2.5 Azide Linkers and 3.2 FITC molecules 14 (1.0 mg, 29 nmole) were dissolved in deuterium oxide (0.741 mL) and placed in a glass microwave reactor vessel. Sodium ascorbate (0.36 mg, 1.8 μmole) and copper sulfate pentahydrate (0.38 mg, 1.5 μmmole) was added to the dendrimer solution. The resulting solution was placed in a microwave reactor for 6.5 minutes at 100 watts with a cut-off temperature of 100° C. The reaction mixture was transferred to an NMR tube and analyzed by NOESY and ¹H NMR spectroscopy using. Lyophilization yielded 2.4 mg of a red solid. ¹H NMR spectrum for 18 is shown in FIG. 6Q.

### Synthesis and Characterization of the Small-Molecule Model System

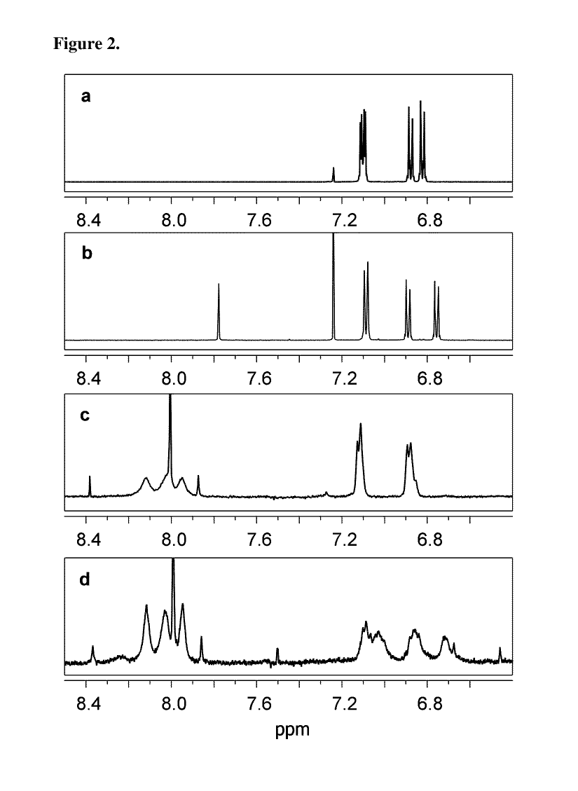

A small molecule model system (4) was first synthesized to facilitate the spectroscopic assignment of triazole-related atoms resulting from successful ‘click’ reactions between dendrimer modules with an azide linker (3c) and dendrimer modules possessing an alkyne linker (2b). This model system utilized the methyl ester forms of the two linkers (2a and 3b). Proton assignments were based upon ¹H NMR and NOESY experiments in CDCl₃. FIG. 1, panel a, displays the cross-peaks for the triazole related protons in the clicked product (4) and Table 1 contains the chemical shifts for these in both the pre- and post-‘click’ reaction states. Protons c, e, and f experience the greatest change in chemical shift as a result of the ‘click’ reaction. Also of interest is the region between 6.4 and 8.5 ppm (FIG. 2, panel a and b), which shows the up-field shift for peak g from 6.85 ppm to 6.78 ppm.

### Synthesis and Characterization of the Model Dendrimer System

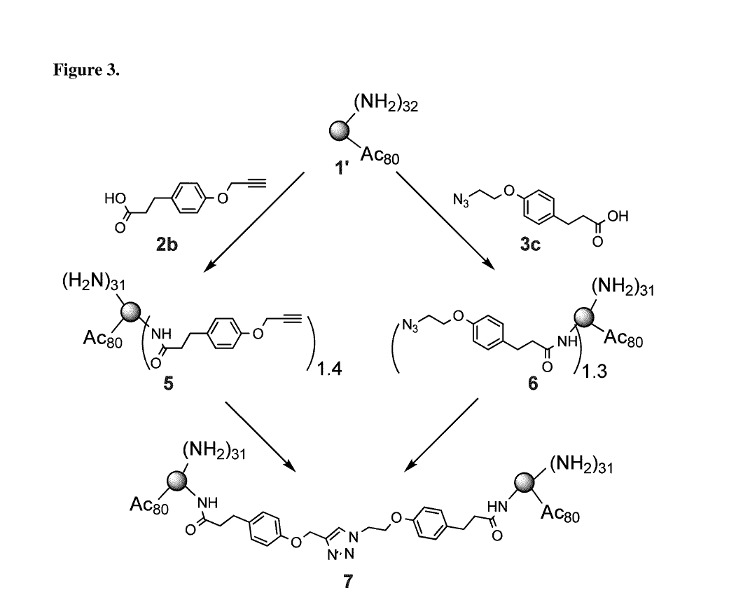

Dendrimers without target or dye functionalities, possessing only the ‘click’ reaction functional groups (5 and 6), were employed to develop ‘click’ reaction conditions (FIG. 3). Because many of the proton peaks associated with the dendrimer-conjugated alkyne and azide linkers (particularly those closest to the ‘click’ reaction sites) overlap in the ¹H NMR spectra with other protons belonging to the PAMAM dendrimer, NOESY experiments were used to document proton chemical shifts via the resolution of cross peaks in the 2-D spectra (FIG. 1, panel b). The chemical shifts for the triazole related protons in the dendrimer system both pre- and post-‘click’ reaction can be found in Table 1. The region between 6.4 and 8.5 ppm in the proton spectra for the pre- and post-'click'ed dendrimer samples can be found in FIG. 2, panel c and d. In the spectra for the pre-'click' reaction mixture (panel c), both sets of aromatic protons overlap at 6.90 ppm (b and g) and 7.13 ppm (a and h). In the sample post-'click' reaction (panel d), the aromatic protons no longer overlap. Protons a and h partially overlap at 7.09 ppm and 7.06 ppm, and protons b and g are found at 6.90 ppm and 6.74 ppm, respectively.

A comparison of the chemical shifts in Table 1 for the small molecule system and the dendrimer system reveals good correlations for both the pre- and post-reaction states. This indicates that a successful ‘click’ reaction has occurred between the azide and alkyne conjugated dendrimers. It is important to note that whereas chemical shifts for the small molecule model system are determined in CDCl₃, the chemical shifts for the dendrimer sample were detected in D₂O. Although the different solvents could influence the proton chemical shifts, this does not appear to be an issue for these particular molecules.

A peak for the single proton in the triazole ring is absent from both the NOESY and 1D experiments. In the small molecule system this peak is found at 7.80 ppm (FIG. 2, panel b). Working with a similar system using PAMAM dendrons that contained the alkyne and azide moieties at the dendron focal point, Lee et. al. found that the triazole proton peak gradually shifted down-field from 7.77 ppm to 7.93 ppm as the generation of the clicked dendrons increased from 1 to 3 (see, e.g., Lee, J. W.; et al., Tetrahedron 2006, 62, (39), 9193-9200; herein incorporated by reference in its entirety). If this downfield change in chemical shift also holds for the generation 5 dendrimer case, the triazole proton would be overlapped by several peaks between 7.80 ppm and 8.20 ppm associated with the dendrimer (FIG. 2, panel d). The NOESY spectra did not expose any cross peaks in this region with other protons in the linkers that are in close proximity to the triazole ring. The cross-peaks associated with the triazole proton appear to be below the intensity required for NMR detection.

### Synthesis and Characterization of the Folic Acid Targeted Dendrimer System

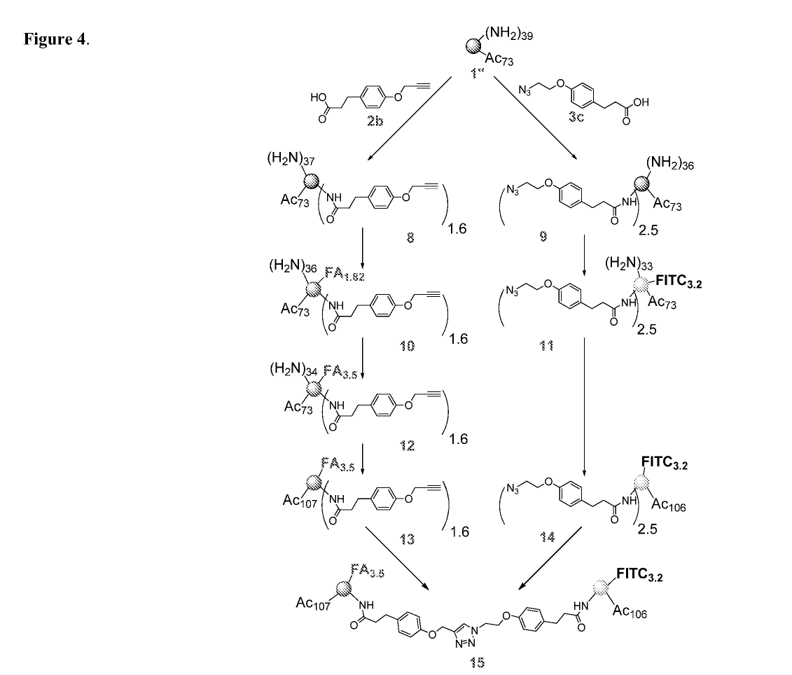

Synthesis of the folic acid targeted modular dendrimer platform is outlined in FIG. 4. Dendrimers with an average of 1.6 alkyne linkers (8) were functionalized with the targeting molecule folic acid. The formation of an amide linkage between one of the remaining primary amines on the dendrimer and one of the carboxylic acid groups in folic acid was achieved by EDC coupling chemistry as previously reported (see, e.g., Majoros, I. J.; et al., Biomacromolecules 2006, 7, (2), 572-579; herein incorporated by reference in its entirety). This reaction conjugated an average of 1.7 folic acid molecules per dendrimer as determined by NMR (10). Because this average was below the optimal range for multi-valent binding (see, e.g., S. Hong, et al., Chem. Biol. 14(1) (2007) 105-113; herein incorporated by reference in its entirety), the reaction was repeated using the dendrimer with an average of 1.6 alkyne linkers and 1.7 folic acid (10). The second reaction resulted in the addition of 1.8 folic acid molecules per dendrimer bringing the final average to 3.5 folic acid molecules per dendrimer (12). The remaining dendrimer primary amines were then fully acetylated to avoid positive charge-based cellular interactions (13) (see, e.g., Hong, S. P., et al., Bioconjugate Chemistry 2004, 15, (4), 774-782; Hong, S. P.; et al., Bioconjugate Chemistry 2006, 17, (3), 728-734; Leroueil, P. R.; et al., Accounts of Chemical Research 2007, 40, (5), 335-342; each herein incorporated by reference in their entireties). Dendrimers with 2.5 azide linkers (9) were functionalized with the dye molecule FITC. Using conditions similar to previously published work (see, e.g., Majoros, I. J.; et al., Journal of Medicinal Chemistry 2005, 48, (19), 5892-5899; herein incorporated by reference in its entirety), an average of 3.2 FITC molecules were coupled to the dendrimer via the formation of a thiourea bond between the primary amine on the dendrimer and the isothiocyanate group in FITC (11). This dendrimer conjugate also was fully acetylated (14). Reverse phase HPLC confirmed that any un-reacted FITC or folic acid molecules had been removed by purification of dendrimer 13 and 14.

The two dendrimer modules (13 and 14) were coupled together using the Cu-catalysed 1,3-dipolar cycloaddition reaction under conditions similar to those used with the model dendrimer system. NOESY experiments provided direct spectroscopic proof that the functionalized dendrimers had been clicked together. Specifically, the AA′BB′ pattern was observed to shift in a fashion identical to that observed for the two model systems previously described (Table 1 and FIG. 2).

In Vitro Testing of the Folic Acid Targeted Dendrimer System with KB Cells

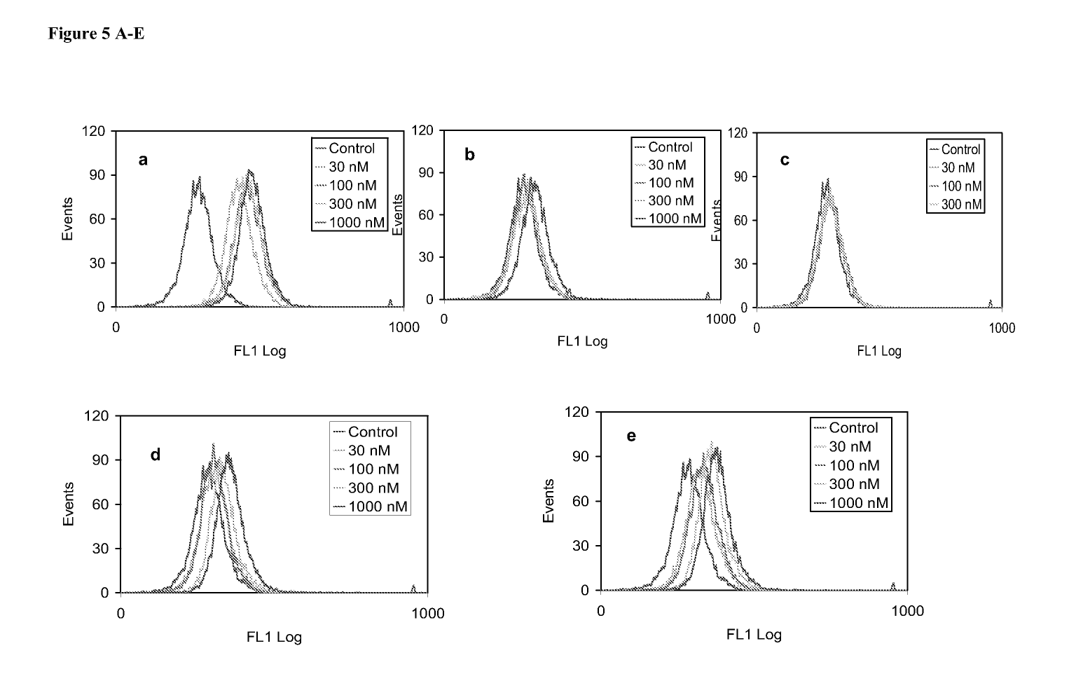

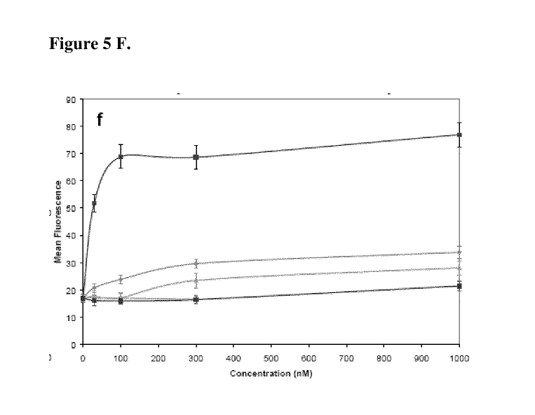

Cellular uptake of the folic acid targeted dendrimer system (15) at four different concentrations (30 nM, 100 nM, 300 nM, and 1000 nM of 15) was measured in KB cells that express a high cellular membrane concentration of folic acid receptor (FAR). Fluorescence uptake was quantified by Flow Cytometry. As seen in FIGS. 5a and 5f-blue, a dose dependent uptake was observed with saturation occurring at 100 nM. This binding affinity is consistent with previous studies on single dendrimer platforms possessing multiple FITC and multiple FA molecules (see, e.g., Thomas, T. P.; et al., Journal of Medicinal Chemistry 2005, 48, (11), 3729-3735; herein incorporated by reference in its entirety).

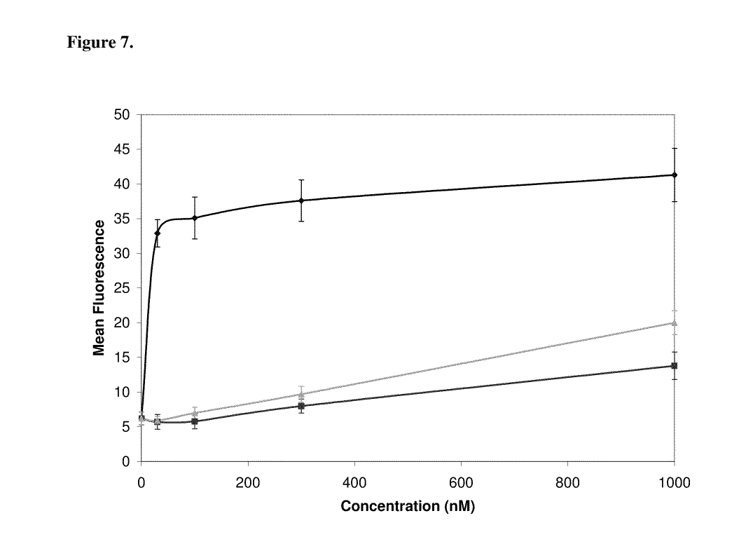

A series of control experiments were performed in order to ensure that uptake of the folic acid targeted dendrimer system (15) was occurring via receptor-mediated endocytosis and not non-specific membrane interactions. The first set of controls measured uptake of single dendrimers possessing the azide linker and multiple FITC (14) at 30 nM, 100 nM, 300 nM, and 1000 nM (FIGS. 5b and 5f-purple). No uptake was observed for this sample above the background level. The second control sample contained a non-conjugated (un-clicked) mixture of the two dendrimers functionalized with either FITC or folic acid (13 and 14). Uptake of this mixture was quantified at 30 nM, 100 nM, and 300 nM (FIGS. 5c and 5f-teal). At all three concentrations, no florescence uptake was observed. This control eliminates the possibility that the dendrimer modules could form a non-covalently linked complex that would be internalized. A third control sample (18) was composed of an un-targeted dendrimer module (17) coupled to the FITC conjugated imaging module (14). The un-targeted dual module platform (18) was assembled under the same conditions used to form the folic acid targeted platform (15). Mean fluorescence uptake of the un-targeted platform (18) is shown in FIG. 7. At concentrations up to 300 nM, no uptake was observed beyond the background level. This un-targeted dendrimer platform control matches the molecular weight and size of the folic acid targeted dendrimer system (15).

The final set of controls investigated active blocking of the folic acid receptor by either free folic acid (FIGS. 5d and 5f-green) or a folic acid-dendrimer conjugate without a fluorescent dye (13) (FIGS. 5e and 5f-orange) to prevent the specific up-take of the folic acid targeted dendrimer systema A 20 fold excess of blocking agent was employed relative to the targeted platform. For the blocking experiment using the single dendrimer-folic acid conjugate (13), molar equivalence was based on the folic acid content of the sample rather than the dendrimer content. Both blocking agents were evaluated at 30 nM, 100 nM, 300 nM, and 1000 nM. Complete blocking is achieved using free folic acid concentrations up to 100 nM. While the dendrimer-folic acid conjugate is not as effective at blocking as the free folic acid, approximately 75% blocking is achieved. These binding data indicate that the cellular association of the folic acid targeted dendrimer system occurs through the folic acid receptor rather than via non-specific interactions. On a more fundamental level, the biological results prove again that the folic acid conjugate dendrimer module is covalently linked by ‘click’ chemistry to dendrimer module functionalized with FITC.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising a first dendrimer coupled with a second dendrimer, wherein said coupling is a covalent attachment between said first dendrimer and said second dendrimer.

2. The composition of claim 1, wherein said covalent attachment is selected from the group consisting of 1) between an alkyne linker on said first dendrimer and an azide linker on said second dendrimer, and 2) between an alkyne linker on said second dendrimer and an azide linker on said first dendrimer.

3. The composition of claim 1, wherein first dendrimer and second dendrimer each independently comprise at least one functional group selected from the group consisting of a therapeutic agent, a targeting agent, a trigger agent, and an imaging agent.

4. The composition of claim 2, wherein said therapeutic agent is selected from the group consisting of a chemotherapeutic agent, an anti-oncogenic agent, an anti-angiogenic agent, a tumor suppressor agent, an anti-microbial agent, an expression construct comprising a nucleic acid encoding a therapeutic protein, a pain relief agent, a pain relief agent antagonist, an agent designed to treat an inflammatory disorder, an agent designed to treat an autoimmune disorder, an agent designed to treat inflammatory bowel disease, and an agent designed to treat inflammatory pelvic disease;
wherein said agent designed to treat an inflammatory disorder is selected from the group consisting of an antirheumatic drug, a biologicals agent, a nonsteroidal anti-inflammatory drug, an analgesic, an immunomodulator, a glucocorticoid, a TNF-α inhibitor, an IL-1 inhibitor, and a metalloprotease inhibitor;
wherein said antirheumatic drug is selected from the group consisting of leflunomide, methotrexate, sulfasalazine, and hydroxychloroquine;
wherein said biologicals agent is selected from the group consisting of rituximab, finfliximab, etanercept, adalimumab, and golimumab;
wherein said nonsteroidal anti-inflammatory drug is selected from the group consisting of ibuprofen, celecoxib, ketoprofen, naproxen, piroxicam, and diclofenac;
wherein said analgesics is selected from the group consisting of acetaminophen, and tramadol;
wherein said immunomodulator is selected from the group consisting of anakinra, and abatacept;
wherein said glucocorticoid is selected from the group consisting of prednisone, and methylprednisone; and
wherein said TNF-α inhibitor is selected from the group consisting of adalimumab, certolizumab pegol, etanercept, golimumab, and infliximab.

5. The composition of claim 4, wherein said autoimmune disorder and/or inflammatory disorder is selected from the group consisting of arthritis, psoriasis, lupus erythematosus, Crohn's disease, and sarcoidosis;
wherein said arthritis is selected from the group consisting of osteoarthritis, rheumatoid arthritis, septic arthritis, gout and pseudo-gout, juvenile idiopathic arthritis, psoriatic arthritis, Still's disease, and ankylosing spondylitis.

6. The composition of claim 3, wherein said first dendrimer and/or said second dendrimer comprise at least one therapeutic agent conjugated with said first dendrimer and/or said second dendrimer via said trigger agent.

7. The composition of claim 3, wherein said trigger agent has a function selected from the group consisting of permitting a delayed release of a functional group from the dendrimer, permitting a constitutive release of the therapeutic agent from the dendrimer, permitting a release of a functional group from the dendrimer under conditions of acidosis, permitting a release of a functional group from a dendrimer under conditions of hypoxia, and permitting a release of the therapeutic agent from a dendrimer in the presence of a brain enzyme.

8. The composition of claim 3, wherein said trigger agent is selected from the group consisting of an ester bond, an amide bond, an ether bond, an indoquinone, a nitroheterocyle, and a nitroimidazole.

9. The composition of claim 3, wherein said imaging agent is selected from the group consisting of fluorescein isothiocyanate (FITC), 6-TAMARA, acridine orange, and cis-parinaric acid.

10. The composition of claim 3, wherein said targeting agent is selected from the group consisting of an agent binding a receptor selected from the group consisting of CFTR, EGFR, estrogen receptor, FGR2, folate receptor, IL-2 receptor, and VEGFR; an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein; an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen; transferrin; and a synthetic tetanus toxin fragment.

11. The composition of claim 1, wherein said first dendrimer and/or said second dendrimer is selected from the group consisting of a polyamideamine (PAMAM) dendrimer, a polypropylamine (POPAM) dendrimer, and a PAMAM-POPAM dendrimer.

12. The composition of claim 1, wherein at least one of said said first dendrimer and/or said second dendrimer is a Baker-Huang PAMAM dendrimer.

13. The method of claim 1, wherein at least one of said said first dendrimer and/or said second dendrimer has a generation between 0 and 5.

14. The composition of claim 1, wherein at least one of said said first dendrimer and/or said second dendrimer is at least partially acetylated.

15. A method of coupling a first dendrimer with a second dendrimer, comprising exposing said first dendrimer to said second dendrimer under conditions such that covalent attachment occurs between an alkyne linker on said first dendrimer and an azide linker on said second dendrimer.

16. The method of claim 15, wherein first dendrimer and second dendrimer each independently comprise at least one functional group selected from the group consisting of a therapeutic agent, an imaging agent, and a targeting agent.

17. The method of claim 15, wherein said first dendrimer and/or said second dendrimer is selected from the group consisting of a polyamideamine (PAMAM) dendrimer, a polypropylamine (POPAM) dendrimer, and a PAMAM-POPAM dendrimer.

18. The method of claim 15, wherein said coupling occurs via a cycloaddition reaction between said first dendrimer and said second dendrimer.

19. A method of treating a disorder selected from the group consisting of osteoarthritis, rheumatoid arthritis, septic arthritis, gout and pseudo-gout, juvenile idiopathic arthritis, psoriatic arthritis, Still's disease, and ankylosing spondylitis, comprising administering to a subject suffering from said disorder a composition of claim 1.

20. The method of claim 19, wherein said composition is co-administered with an agent selected from the group consisting of an antirheumatic drug, a biologicals agent, a nonsteroidal anti-inflammatory drug, an analgesic, an immunomodulator, a glucocorticoid, a TNF-α inhibitor, an IL-1 inhibitor, and a metalloprotease inhibitor;
wherein said antirheumatic drug is selected from the group consisting of leflunomide, methotrexate, sulfasalazine, and hydroxychloroquine;
wherein said biologicals agent is selected from the group consisting of rituximab, finfliximab, etanercept, adalimumab, and golimumab;
wherein said nonsteroidal anti-inflammatory drug is selected from the group consisting of ibuprofen, celecoxib, ketoprofen, naproxen, piroxicam, and diclofenac;
wherein said analgesics is selected from the group consisting of acetaminophen, and tramadol;
wherein said immunomodulator is selected from the group consisting of anakinra, and abatacept;
wherein said glucocorticoid is selected from the group consisting of prednisone, and methylprednisone; and
wherein said TNF-α inhibitor is selected from the group consisting of adalimumab, certolizumab pegol, etanercept, golimumab, and infliximab.


SEQUENCE LISTING (2 sequences)

SEQ ID NO: 1 - protein, 12 aa, Artificial Sequence
  Synthetic
HLNILSTLWKYR

SEQ ID NO: 2 - protein, 7 aa, Artificial Sequence
  Synthetic
YEVDGWK